(19) United States
(12) Patent Application Publication
(10) Pub. No.: US 2018/0243392 A1
(43) Pub. Date: Aug. 30, 2018

(54) CS21 AND LNGA PROTEIN VACCINES

(71) Applicant: VANDERBILT UNIVERSITY, Nashville, TN (US)

(72) Inventors: Oscar G. GOMEZ DUARTE, Clarence Center, NY (US); Chengxian ZHANG, Nashville, TN (US)

(21) Appl. No.: 15/558,761

(22) PCT Filed: Mar. 17, 2016
(86) PCT No.: PCT/US2016/022851

§ 371 Date: Sep. 15, 2017

Related U.S. Application Data
(60) Provisional application No. 62/290,060, filed on Feb. 2, 2016.
(60) Provisional application No. 62/134,026, filed on Mar. 17, 2015.

(51) Int. Cl. A61K 39/108; A61K 39/39; C07K 16/12; A61K 39/40; A61K 45/06; A61P 31/04

(52) U.S. Cl. CPC A61K 39/0258; A61K 39/39; C07K 16/1232; A61K 2039/55544; A61K 45/06; A61P 31/04; A61K 39/40

(57) ABSTRACT

A vaccine composition including an LngA peptide or a fragment or variant thereof and a pharmaceutically acceptable carrier is described. The vaccine composition can be administered to prevent or inhibit an enterotoxigenic Escherichia coli infection in a subject.

### CONTINUING APPLICATION DATA

This application claims the benefit of U.S. Provisional Application Ser. No. 62/134,026 filed Mar. 17, 2015, and U.S. Provisional Application Ser. No. 62/290,060, filed on Feb. 2, 2016, both of which are incorporated by reference herein.

### GOVERNMENT FUNDING

The present invention was made with Government support under Grant No. AI 079410 from the National Institute of Health. The Government has certain rights in the invention.

### SEQUENCE LISTING

The instant application contains a Sequence Listing which has been submitted in ASCII format via EFS-Web and is hereby incorporated by reference in its entirety. Said ASCII copy, created on Mar. 17, 2016, is named VU-024063WOORD_SL.txt and is 42,878 bytes in size.

### BACKGROUND

Enterotoxigenic Escherichia coli (ETEC) are a leading cause of traveler's diarrhea and childhood morbidity and mortality in low-income countries and there is no vaccine available for ETEC diarrhea prevention at this time. Kotloff et al., The Lancet. 382(9888):209-22 (2013). ETEC gut colonization by coli surface antigens (CSs) is crucial for pathogenicity and CS21 (a.k.a. longus for long pilus), is one the most prevalent CSs among worldwide ETECs. CS21, toxin-co-regulated pilus (TCP) of Vibrio cholerae, bundle-forming pilus (BFP) of enteropathogenic E. coli and CS8 (CFA/III) of ETEC are class-B type-4 pili. Girón et al., Gene. 192(1):39-43 (1997). A 16-gene cluster, essential in the biosynthesis of CS21, includes structural and regulatory genes. Gómez-Duarte et al., J. Bacteriol. 189(24):9145-49 (2007). The CS21 structural subunit gene lngA encodes a 22 kDa mature protein. Polymerization of thousands of LngA proteins by the bacterial membrane-bound CS21 assembly apparatus results in the CS21 three-dimensional pilus filament. LngA is implicated in CS21-mediated ETEC specific adherence to intestinal cells. Guevara et al. Microbiol. Read. Engl. 159(Pt 8):1725-35 (2013). CS21 also mediated bacterial self-aggregation believed to promote resistance to environmental stress factors (including antimicrobials) Cruz-Córdova et al. Front. Microbiol. 5:709 (2014).

Latin America and the Caribbean as well as the Middle East and North African geographic regions have the highest prevalence of CS21 positive ETEC clinical isolates. Among Latin American countries, Chile has the highest proportion of CS21 positive ETEC strains with a prevalence ranging from 38 to 71.8%. The prevalence of CS21 positive ETECs in Colombia, Brazil, Bolivia and Argentina was 50%, 32.8%, 23.2%, and 17%, respectively. The prevalence of CS21 positive ETECs in African and Asian countries including Egypt, South Korea and Bangladesh was 15%, 19.9%, and 19%, respectively. Spanish travelers with diarrhea reported CS21 in 58% of ETEC isolates. The high prevalence of CS21 among ETEC clinical isolates globally makes this colonization factor an important antigen to be included in a polyvalent vaccine against ETEC diarrhea.

ETEC CSs are highly immunogenic and are promising components of a multivalent ETEC vaccine. Sjöling et al., Expert Rev. Vaccines. 14(4):551-60 (2015); Svennerholm A-M, Tobias J., Expert Rev. Vaccines. 7(6):795-804 (2008). Unfortunately, knowledge on CS21 immunogenicity and immunoprotection is limited and none of the previously described ETEC vaccines have included CS21 antigens. Consequently, it is imperative to investigate the vaccine potential of CS21 with respect to immunogenicity and protection against ETEC intestinal colonization.

### SUMMARY

The inventors hypothesized that CS21 protein polymer and its LngA subunit are highly immunogenic and able to induce specific anti-CS21 and anti-LngA antibodies. Anti-LngA antibody binding to CS21 inhibits CS21-mediated ETEC adherence to intestinal epithelial cells and protects against ETEC intestinal colonization. Subsequently, they demonstrated that CS21 and LngA antigen delivery to a mammalian host by different routes may induce specific and protective immune responses. They have shown that both LngA and CS21 are highly immunogenic and that CS21 immunogen can inhibit ETEC colonization. Furthermore, anti-CS21 and anti-LngA antibodies recognize CS8, another important ETEC colonization factor similar to CS21, and these antibodies may inhibit the CS8-mediated interactions to intestinal cells. Their studies on immunogenicity and immnunoprotection place CS21 in the list of suitable vaccine candidates for a future multivalent vaccine against ETEC diarrhea.

### BRIEF DESCRIPTION OF THE FIGURES

FIGS. 1A and 1B provide protein sequence alignment of 19 LngA variants (SEQ ID NOS 8-26, respectively, in order of appearance) (A) and type IV pili major subunits LngA (SEQ ID NO: 6), CofA (SEQ ID NO: 28), BfpA (SEQ ID NO: 29), and TcpA (SEQ ID NO: 27) (B). Two major genetic variation regions in LngA are shown, which span from residues 56 to 77 and 148 to 197. Two disulfide-bond-forming cysteines in the major subunits of type-4 pili are indicated by arrows. Five potential surface accessible B cell epitopes were identified at residues ⁵⁰AYQRDGKYPDFV⁶¹ (SEQ ID NO: 1), ⁷⁰TIKTDTSGIP⁷⁹ (SEQ ID NO: 2), ⁸⁹ITPDEVRNN⁹⁷ (SEQ ID NO: 3), ¹⁰⁹LTSNGAQVK¹¹⁷ (SEQ ID NO: 4), and ¹⁷⁸GNNGQTTLT¹⁸⁶ (SEQ ID NO: 5), tentatively designated as Epitope 1 to 5 (EP1 to EPS).

FIGS. 2A-2C provide graphs showing the potential linear B cell epitopes of LngA major subunit derived from strain E9034A predicted by Bepipred Linear Epitope Prediction (A), Parker Hydrophilicity Prediction (B), and Emini Surface Accessibility Prediction (C) of Antibody Epitope Prediction on IEDB Analysis Resources. Five potential B cell epitopes are tentatively designated as EP1-5.

FIG. 3 provides images showing that LngA-recombinant and CS21 pili antigens recognized by monoclonal anti-LngA and polyclonal murine sera. Panel A. LngA-his tag recombinant and CS21 purified native pili preparation were separated in an SDS-PAGE gel. The Coomassie blue stained gel is shown on the left and the Western blot labeled with anti-LngA monoclonal antibody is shown on the right. CS21 pili preparations were derived from E9034A ETEC strain; LngA-his tag protein was obtained from Genscript; BL21 E. coli was used as negative control; and E9034AΔlngA pili preparation was used as LngA negative control. Panel B. Western blot of CS21 and CS8 pili from ETEC strains from different geographic locations using murine anti-LngA polyclonal sera. Purified CS21 and CS8 pili from ETEC strains originated in different countries were evaluated by Western blot using two different antisera, one derived from a mouse immunized IP with LngA-his tag antigen plus IFA and the second obtained from a mouse immunized IP with CS21 pili antigen plus IFA. Pili preparations from E. coli DH5a E. coli BL21 and CS21⁺ ETEC E9034A strains were used as negative and positive controls and LngA-His recombinant protein was used as LngA peptide control. The size of LngA is 22 kDa.

FIGS. 4A-4D provide graphs showing anti-LngA antibody responses in mice immunized with LngA recombinant protein. Mice were immunized with LngA plus IFA, LngA plus ALUM, or LngA plus CT via intraperitoneal routes three times at two-week intervals. Controls were mock immunized with PBS+IFA or PBS+ALUM. The anti-LngA serum IgG (A), fecal IgA (B), and intestine wash IgA (C) were titrated by ELISA. The anti-LngA serum IgG responses increased steadily overtime until reaching a peak response at 37 days after the primary immunization (D). The closed circles are the control mice and the open circles are the immunized mice. The asterisk (*) indicates p-value <0.05.

FIGS. 5A-5D provide graphs showing anti-LngA and anti-CT antibody responses in mice immunized with CS21 pili antigen and CT adjuvant. Mice were immunized with CS21 plus ALUM via subcutaneous injection (SC), CS21 plus CT via intranasal route (IN), CS21 plus IFA via intraperitoneal route (IP), or CS21 plus ALUM. via IP route three times at a two-week intervals. Controls were mock immunized with PBS plus corresponding adjuvants. Naïve mice were not immunized nor challenged. The anti-LngA serum IgG (A), fecal IgA (B), intestine wash IgA (C) and anti-CT antibody (D) were titrated by ELISA. The closed circles are the control mice and the open circles are the immunized mice. The asterisk indicates p-values <0.05.

FIGS. 6A-6E provide graphs showing the effect of LngA and CS21 immunization on gut colonization by measurement of bacterial shedding from mouse feces. To evaluate the role of CS21 and LngA in protection against gut colonization by ETEC, bacterial shedding was measured from mice feces collected daily by quantification of ETEC colony-forming units per gram of feces. Panel A. Mice immunized with LngA+IFA or LngA+CT via intraperitoneal (IP) route. Panel B. CS21+CT via the intranasal route (IN). Panel C, CS21+ALUM via the subcutaneous route (SC). Panel D, CS21+IFA via IP route. Panel E, CS21+ALUM via IP route. All mice except the naïve group were orally challenged with CS21⁺ ETEC E9034A strain. Mice feces were collected and suspended in sterile PBS and the bacteria CFU per gram of feces was determined by plating feces suspension on MacConkey plates. The asterisk (*) indicates a p-value <0.05.

FIG. 7 provides an amino acid sequence for the LngA protein.

### DETAILED DESCRIPTION

The present invention provides a vaccine composition including an LngA peptide or a fragment or variant thereof and a pharmaceutically acceptable carrier is described. The vaccine composition can be administered to prevent or inhibit an enterotoxigenic Escherichia coli infection in a subject.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, and other references mentioned herein are incorporated by reference in their entirety.

### Definitions

As used in the description of the invention and the appended claims, the singular forms “a,” “an,” and “the” are intended to include the plural forms as well, unless the context clearly indicates otherwise. In addition, the recitations of numerical ranges by endpoints include all numbers subsumed within that range (e.g., 1 to 5 includes 1, 1.5, 2, 2.75, 3, 3.80, 4, 5, etc.).

As used herein, the term “polypeptide” is intended to encompass a singular “polypeptide” as well as plural “polypeptides,” and comprises any chain or chains of two or more amino acids. Thus, as used herein, terms including, but not limited to “peptide,” “dipeptide,” “tripeptide,” “protein,” “amino acid chain,” or any other term used to refer to a chain or chains of two or more amino acids, are included in the definition of a “polypeptide,” and the term “polypeptide” may be used instead of, or interchangeably with any of these terms. The term further includes polypeptides which have undergone post-translational modifications, for example, glycosylation, acetylation, phosphorylation, amidation, derivatization by known protecting/blocking groups, proteolytic cleavage, or modification by non-naturally occurring amino acids.

The term “polynucleotide” is intended to encompass a single nucleic acid or nucleic acid fragment as well as plural nucleic acids or nucleic acid fragments, and refers to an isolated molecule or construct, e.g., a bacterial genome, messenger RNA (mRNA), plasmid DNA (pDNA), or derivatives of pDNA (e.g., minicircles as described in (Darquet, A-M et al., Gene Therapy 4:1341-1349 (1997)) comprising a polynucleotide. A polynucleotide may be provided in linear (e.g., mRNA), circular (e.g., plasmid), or branched form as well as double-stranded or single-stranded forms. A polynucleotide may comprise a conventional phosphodiester bond or a non-conventional bond (e.g., an amide bond, such as found in peptide nucleic acids (PNA)).

The term “antigen” as used herein refer to a portion or portions of molecules which are capable of inducing a specific immune response in a subject alone or in combination with an adjuvant. The term “epitope,” as used herein, refers to a portion of a polypeptide having antigenic or immunogenic activity in an animal, for example a mammal, for example, a human.

The term “immune response”, as used herein, refers to an alteration in the reactivity of the immune system of an animal in response to an antigen or antigenic material and may involve antibody production, induction of cell-mediated immunity, complement activation, development of immunological tolerance, or a combination thereof.

The term “passive immunity” refers to the immunity to an antigen developed by a host animal, the host animal being given antibodies produced by another animal, rather than producing its own antibodies to the antigen. The term “active immunity” refers to the production of an antibody by a host animal as a result of the presence of the target antigen.

The term “immunoprotection” as used herein, mean an immune response that is directed against one or more antigen so as to protect against disease and/or infection by a pathogen in a vaccinated animal. For purposes of the present invention, protection against disease includes not only the absolute prevention of the disease, but also any detectable reduction in the degree or rate of disease, or any detectable reduction in the severity of the disease or any symptom in the vaccinated animal as compared to an unvaccinated infected or diseased animal, which is also referred to as inhibition of the disease. Immunoprotection can be the result of one or more mechanisms, including humoral and/or cellular immunity.

The term “vaccine”, as used herein, refers to a preparation that is used to establish immunity to a disease, thereby protecting a body from a disease, or reducing the chances of a body becoming affected by the disease. Vaccines can be preventative against the effects of a future infection or therapeutic (intended to reduce the severity of an infection or a disease, typically by assisting the immune system in fighting the infection or disease). In certain embodiments of the invention, a vaccine is a preparation that is used to establish immunity to a disease in the offspring of the individual to which the vaccine is delivered.

“Treating”, as used herein, means ameliorating the effects of, or delaying, halting or reversing the progress of a disease or disorder. The word encompasses reducing the severity of a symptom of a disease or disorder and/or the frequency of a symptom of a disease or disorder.

A “subject”, as used therein, can be a human or non-human animal Non-human animals include, for example, livestock and pets, such as ovine, bovine, porcine, canine, feline and murine mammals, as well as reptiles, birds and fish. Preferably, the subject is human.

The language “effective amount” or “therapeutically effective amount” refers to a nontoxic but sufficient amount of the composition used in the practice of the invention that is effective to provide effective vaccination or treatment in a subject. That result can be reduction and/or alleviation of the signs, symptoms, or causes of a disease or disorder, or any other desired alteration of a biological system. An appropriate effective amount for a subject may be determined by one of ordinary skill in the art using routine experimentation.

A “prophylactic” or “preventive” treatment is a treatment administered to a subject who does not exhibit signs of a disease or disorder, or exhibits only early signs of the disease or disorder, for the purpose of decreasing the risk of developing pathology associated with the disease or disorder. Use of a vaccine in a preventive treatment provides immunoprotection.

“Pharmaceutically acceptable carrier” refers herein to a composition suitable for delivering an active pharmaceutical ingredient, such as the vaccine composition of the present invention, to a subject without excessive toxicity or other complications while maintaining the biological activity of the active pharmaceutical ingredient. Protein-stabilizing excipients, such as mannitol, sucrose, polysorbate-80 and phosphate buffers, are typically found in such carriers, although the carriers should not be construed as being limited only to these compounds.

### LngA Antigens

In one aspect, the present invention provides a vaccine composition comprising an LngA peptide or a fragment or variant thereof and a pharmaceutically acceptable carrier. The LngA peptide, also referred to as Longus, is a protein forming the C21 pilus that is used by enterotoxigenic Escherichia coli for gut colonization. The amino acid sequence of the LngA protein is known. See Gomez-Duarte et al., Microbiology, 145 (Pt 7):1809-16 (1999), the disclosure of which is incorporated by reference herein. The mature full-length LngA protein has 206 amino acids and the sequence derived from the wild type E. coli E9034A strain (SEQ ID NO: 6) shown in FIG. 7. In one embodiment of the invention, the LngA peptide is a fragment, variant, derivative, or analogue thereof, e.g., a polypeptide comprising an amino acid sequence at least 80%, 85%, 90%, 95%, 96%, 97%, 98%, 99%, or 100% identical to a known LngA amino acid sequence, e.g., SEQ ID NO: 6, wherein the polypeptide is recognizable by an antibody specifically binds to the LngA sequence.

In some embodiments, the vaccine composition comprises a CS21 pilus or a substantial fragment thereof. A pilus is a hairlike appendage found on the surface of bacteria such as enterotoxigenic Escherichia coli. The CS21 pilus is a type IV pilus, which can generate motile force. The CS21 pilus comprises multiple LngA proteins. A substantial fragment, as the term is used herein, is a CS21 pilus including a plurality of LngA proteins.

An LngA peptide fragment or variant thereof as used herein can comprise an epitope located in the LngA protein, wherein the LngA peptide comprises, consists essentially of, or consists of about 5-126 amino acids, about 5-75 amino acids, about 5-50 amino acids, about 5-25 amino acids, about 5-20 amino acids, about 5-15 amino acids, about 8-15 amino acids, or about 9-12 amino acids. Non-limiting examples of LngA antibody epitopes comprise, consists essentially of, or consists of an amino acid sequence selected from the group consisting of AYQRDGKYPDFV (SEQ ID NO: 1; amino acids 50-61 of LngA), TIKTDTSGIP (SEQ ID NO: 2; amino acids 70-79 of LngA), ITPDEVRNN (SEQ ID NO: 3; amino acids 89-97 of LngA), LTSNGAQVK (SEQ ID NO: 4; amino acids 109-117 of LngA), and GNNGQTTLT (SEQ ID NO: 5; amino acids 178-186 of LngA).

Alternatively, LngA peptides can be variants thereof, which are recognizable by an antibody that specifically binds to a peptide consisting of SEQ ID NOs: 1, 2, 3, 4, 5, or 6. For example, in some embodiments, the variant of the LngA peptide is at least 80%, 85%, 90%, 95%, 96%, 97%, 98%, 99%, or 100% identical to SEQ ID NOs: 1, 2, 3, 4, 5, or 6, wherein the variants are recognizable by an antibody specifically binds to a peptide consisting of SEQ ID NO: 1, 2, 3, 4, 5, or 6.

The terms “fragment” or “variant” when referring to an LngA polypeptide includes any polypeptides which retain at least some of the immunogenicity or antigenicity of the naturally-occurring LngA or CofA proteins. Fragments of LngA polypeptides of the present invention include proteolytic fragments, deletion fragments and in particular, fragments of polypeptides which exhibit increased solubility during expression, purification, and or administration to an animal. Fragments of LngA polypeptides further include proteolytic fragments or deletion fragments which exhibit reduced pathogenicity when delivered to a subject. Polypeptide fragments further include any portion of the polypeptide which comprises an antigenic or immunogenic epitope of the native polypeptide, including linear as well as three-dimensional epitopes.

An “epitopic fragment” of a polypeptide antigen is a portion of the antigen that contains an epitope. An “epitopic fragment” may, but need not, contain amino acid sequence in addition to one or more epitopes.

The term “variant,” as used herein, refers to a polypeptide that differs from the recited polypeptide due to amino acid substitutions, deletions, insertions, and/or modifications. Variants may occur naturally, such as a subtypic variant. The term “subtypic variant” is intended polypeptides or polynucleotides that are present in a different enterotoxigenic Escherichia coli subtypes including, but not limited to, LngA and CS21 from E9038A strain (GeneBank ID: NG_036490.1); LngA and CS21 Group 1 variants (From ETEC strains: B2C, GeneBank ID: EU107090.1; strain 01117-5, GenBank ID: EU107089.1; strain M145-C2, GenBank ID: EU107091.1; strain M424-C1, GenBank ID: EU107092.1; strain P307, GenBank ID: EU107093.1; strain M408, GenBank ID: EU107094.1; strain M526-C6B, GenBank ID: EU107107.1), LngA and CS21 Group 2 variants (From ETEC strain 10159a, GenBank ID: EU107095.1; ETEC strain 11381a, GenBank ID: EU107096.1; ETEC strain 2108-2, GenBank: EU107097.1; ETEC strain B7A, GenBank ID: EU107098.1; ETEC strain G1026, GenBank ID: EU107099.1; ETEC strain M445-C1, GenBank ID: EU107100.1; ETEC strain M452-C1, GenBank ID: EU107101.1; ETEC strain M626-C, GenBank ID: EU107102.1; ETEC strain M633-C1, GenBank ID: EU107103.1; ETEC strain MP215-1, GenBank ID: EU107104.1; ETEC strain BR5, GenBank ID: EU107105.1); LngA Group 3 variants (From strains: ECOR27), CofA and CS8 Group variant 1 (ETEC strain 2528C and 260-1), CofA and CS8 Group variant 2 (ETEC strain M403-C1), LngB variants from Groups 1, Groups 2.

Non-naturally occurring variants may be produced using art-known mutagenesis techniques. In one embodiment, variant polypeptides differ from an identified sequence by substitution, deletion, or addition of five amino acids or fewer. Such variants may generally be identified by modifying a polypeptide sequence, and evaluating the antigenic properties of the modified polypeptide using, for example, the representative procedures described herein.

Polypeptide variants exhibit at least about 80-90%, for example, 80%, 85%, 90%, 94%, 95%, 96%, 97%, 98%, 99%, or 99.9% sequence identity with identified polypeptides. Variant polypeptides may comprise conservative or non-conservative amino acid substitutions, deletions or additions. Peptide variants also include derivatives and analogs. Derivatives of polypeptides are polypeptides which have been altered so as to exhibit additional features not found on the native polypeptide. Examples include fusion proteins. An analog is another form of a polypeptide of the present invention. An example is a proprotein which can be activated by cleavage of the proprotein to produce an active mature polypeptide.

Variants may also, or alternatively, contain other modifications, whereby, for example, a polypeptide may be conjugated or coupled, e.g., fused to an additional polypeptide, e.g., a signal (or leader) sequence at the N-terminal end of the protein which co-translationally or post-translationally directs transfer of the protein. The polypeptide may also be conjugated or produced coupled to a linker or other sequence for ease of synthesis, purification or identification of the polypeptide (e.g., 6-His (SEQ ID NO: 7)), or to enhance binding of the polypeptide to a solid support. For example, a polypeptide may be conjugated or coupled to an immunoglobulin Fc region. The polypeptide may also be conjugated or coupled to a sequence that imparts or modulates the immune response to the polypeptide (e.g., a T-cell epitope, B-cell epitope, cytokine, chemokine, etc.) and/or enhances uptake and/or processing of the polypeptide by antigen presenting cells or other immune system cells. The polypeptide may also be conjugated or coupled to other polypeptides/epitopes from ETEC bacteria and/or from other bacteria and/or other viruses to generate a hybrid immunogenic protein that alone or in combination with various adjuvants can elicit protective immunity to other pathogenic organisms.

The term “sequence identity” as used herein refers to a relationship between two or more polynucleotide sequences or between two or more polypeptide sequences. When a position in one sequence is occupied by the same nucleic acid base or amino acid residue in the corresponding position of the comparator sequence, the sequences are said to be “identical” at that position. The percentage “sequence identity” is calculated by determining the number of positions at which the identical nucleic acid base or amino acid residue occurs in both sequences to yield the number of “identical” positions. The number of “identical” positions is then divided by the total number of positions in the comparison window and multiplied by 100 to yield the percentage of “sequence identity.” Percentage of “sequence identity” is determined by comparing two optimally aligned sequences over a comparison window. In order to optimally align sequences for comparison, the portion of a polynucleotide or polypeptide sequence in the comparison window may comprise additions or deletions termed gaps while the reference sequence is kept constant. An optimal alignment is that alignment which, even with gaps, produces the greatest possible number of “identical” positions between the reference and comparator sequences. The terms “sequence identity” and “identical” are used interchangeably herein. Accordingly, sequences sharing a percentage of “sequence identity” are understood to be that same percentage “identical.” Percentage “sequence identity” between two sequences can be determined using the version of the program “BLAST 2 Sequences” which was available from the National Center for Biotechnology Information as of Sep. 1, 2004, which program incorporates the programs BLASTN (for nucleotide sequence comparison) and BLASTP (for polypeptide sequence comparison), which programs are based on the algorithm of Karlin and Altschul (Proc. Natl. Acad. Sci. USA 90(12):5873-5877, 1993). When utilizing “BLAST 2 Sequences,” parameters that were default parameters as of Sep. 1, 2004, can be used for word size (3), open gap penalty (11), extension gap penalty (1), gap dropoff (50), expect value (10), and any other required parameter including but not limited to matrix option.

Peptides of this invention may be characterized by immunological measurements including, without limitation, Western blot, macromolecular mass determinations by biophysical determinations, such as SDS-PAGE/staining, high pressure liquid chromatography (HPLC) and the like, antibody recognition assays, T-cell recognition assays, major histocompatibility complex (MHC) binding assays, and assays to infer immune protection or immune pathology by adoptive transfer of cells, proteins or antibodies.

### Preparation of LngA Peptides

The CS21 pilus peptides of this invention (as well their naturally occurring variants) may be isolated in the form of a complete intact polymer, or as an LngA subunit, or a polypeptide or fragment thereof. In one embodiment, CS21 is isolated grown in cultured enterotoxigenic Escherichia coli culture, and then purified using centrifugation as described in Example 1 herein. LngA peptides can be further purified using immunoblot procedures according to their molecular mass. Such isolation provides the antigen in a form substantially free from other proteinaceous and non-proteinaceous materials of the microorganism. The molecules comprising the polypeptides and antigens of this invention may be isolated and further purified using any of a variety of conventional methods including, but not limited to: liquid chromatography such as normal or reverse phase, using HPLC, FPLC and the like; affinity chromatography (such as with inorganic ligands or monoclonal antibodies); size exclusion chromatography; immobilized metal chelate chromatography; gel electrophoresis; and the like. One of skill in the art may select the most appropriate isolation and purification techniques without departing from the scope of this invention.

LngA antigens can also be chemically synthesized, for example, using solid phase techniques. See, e.g., Merrifield, J. Am. Chem. Soc. 85, 2149 54, 1963; Roberge et al., Science 269, 202 04, 1995. Protein synthesis can be performed using manual techniques or by automation. Automated synthesis can be achieved, for example, using Applied Biosystems 431A Peptide Synthesizer (Perkin Elmer). Optionally, fragments of an LngA antigen can be separately synthesized and combined using chemical methods to produce a full-length molecule.

Alternatively, the amino acid sequences of the proteins of this invention may be produced recombinantly following conventional genetic engineering techniques. To produce recombinant LngA peptides of this invention, the DNA sequences encoding LngA are inserted into a suitable expression system. The LngA nucleotide sequence is known. See accession number EU107107, and Gomez-Duarte et al., J Bacteriol., 189(24):9145-9 (2007), the disclosure of which is incorporated herein by reference. A recombinant molecule or vector is constructed in which the polynucleotide sequence encoding the selected protein, e.g., LngA, is operably linked to a heterologous expression control sequence permitting expression of the protein. Numerous types of appropriate expression vectors are known in the art for protein expression, by standard molecular biology techniques. Such vectors are selected from among conventional vector types including insects, e.g., baculovirus expression, or yeast, fungal, bacterial or viral expression systems. Other appropriate expression vectors, of which numerous types are known in the art, can also be used for this purpose. Methods for obtaining such expression vectors are well-known. See, Sambrook et al, Molecular Cloning. A Laboratory Manual, 2d edition, Cold Spring Harbor Laboratory, New York (1989); Miller et al, Genetic Engineering, 8:277-298 (Plenum Press 1986) and references cited therein.

Suitable host cells or cell lines for transformation by this method include bacterial cells. For example, the various strains of E. coli, e.g., HB101, MC1061, and strains used in the following examples, are well-known as host cells in the field of biotechnology. Various strains of E. coli, B. subtilis, Pseudomonas, Streptomyces, other bacilli, or yeast and the like are also employed in this method.

Thus, the present invention provides a method for producing recombinant LngA proteins, which involves transformation, e.g., by conventional means such as electroporation, a host cell with at least one expression vector containing a polynucleotide of the invention under the control of a transcriptional regulatory sequence. The transfected or transformed host cell is then cultured under conditions that allow expression of the protein. The expressed protein is recovered, isolated, and optionally purified from the cell (or from the culture medium, if expressed extracellularly) by appropriate means known to one of skill in the art.

For example, the proteins are isolated in soluble form following cell lysis, or extracted using known techniques, e.g., in guanidine chloride. If desired, the proteins or fragments of the invention are produced as a fusion protein. Such fusion proteins are those described above. Alternatively, for example, it may be desirable to produce fusion proteins to enhance expression of the protein in a selected host cell, to improve purification, or for use in monitoring the presence of LngA in tissues, cells or cell extracts. Suitable fusion partners for the proteins of the invention are well known to those of skill in the art and include, among others, β-galactosidase, glutathione-S-transferase, poly-histidine and maltose binding protein.

Prophylactic Treatment with LngA Vaccine Compositions

Another aspect of the invention provides a method of preventing or inhibiting an enterotoxigenic Escherichia coli (ETEC) infection in a subject by administering a vaccine composition comprising an LngA peptide or a fragment or variant thereof and a pharmaceutically acceptable carrier to the subject. In some embodiments, the invention is directed to a method of inducing an immune response against enterotoxigenic Escherichia coli in a subject, e.g., a human comprising administering an effective amount of a vaccine composition comprising an LngA peptide or a fragment or variant thereof and a pharmaceutically acceptable carrier to the subject.

The method of preventing or inhibiting an ETEC infection can include administering a vaccine composition comprising any of the LngA peptides or effective fragments or variants thereof described herein. In some embodiments, LngA antibody epitopes comprise, consists essentially of, or consists of an amino acid sequence selected from the group consisting of AYQRDGKYPDFV (SEQ ID NO: 1), TIKTDTSGIP (SEQ ID NO: 2), ITPDEVRNN (SEQ ID NO: 3), LTSNGAQVK (SEQ ID NO: 4), and GNNGQTTLT (SEQ ID NO: 5). In further embodiments, the vaccine composition includes a variant of the LngA peptide is at least 80%, 85%, 90%, 95%, 96%, 97%, 98%, 99%, or 100% identical to SEQ ID NOs: 1, 2, 3, 4, 5, or 6, wherein the variants are recognizable by an antibody specifically binds to a peptide consisting of SEQ ID NO: 1, 2, 3, 4, 5, or 6.

E. coli is an important bacterial species in the human alimentary tract, and most species are harmless commensals that live in a symbiotic relationship with the host. However, some E. coli encode one or more virulence factors that can render them pathogenic. ETEC possess two major virulence factors; specific adherence appendices called fimbriae that mediate adherence to the host epithelium, and entoerotoxins responsible for fluid homeostasis perturbation. A large number of enterotoxigenic E. coli strains have been identified, and their role in gastrointestinal tract infection and diarrhea has been extensively studied. Dubreuil, J. D., Curr Issues Mol Biol. 14(2):71-82 (2012).

The present invention provides methods of administering a vaccine composition comprising LngA or an effective fragment or variant thereof and a pharmaceutically acceptable carrier to a subject. In some embodiments of the invention, such methods confer immunoprotection to the individual to which the composition is administered. In other embodiments of the invention, immunity is conferred to one or more offspring of the individual to which the composition is administered. For example, the individual being administered the composition can be a pregnant female, whose future or current offspring benefit from immune protection. Such immunity may be passed from mother to child, for example, through breastmilk and/or through blood exchanged between from mother and fetus via the placenta.

In some embodiments of the invention, an effective amount of a vaccine composition of the invention produces an elevation of antibody titer to at least two or three times the antibody titer prior to administration.

As used herein, “a subject in need of therapeutic and/or preventative immunity” refers to an animal which it is desirable to prophylactically treat, i.e., to prevent, cure, retard, or reduce the severity of symptoms related to an enterotoxigenic Escherichia coli infection, and/or result in no worsening of the symptoms over a specified period of time.

In some embodiments, prophylactic treatment is provided to subjects susceptible (or predisposed) to developing chronic infection for preventing the development of acute or chronic infection. Where the susceptible individuals are identified prior to or during infection, for instance, as described herein, the composition can be targeted to them, minimizing need for administration to a larger population.

In some embodiments, the LngA peptide or a fragment or variant thereof is expressed by a live recombinant bacterial vector. Bacterial vector vaccines are well-known to those skilled in the art. See Curtiss, In: New Generation Vaccines: The Molecular Approach, Ed., Marcel Dekker, Inc., New York, N.Y., pages 161-188 and 269-288 (1989). These vaccines can be administered to the host (e.g., orally, intranasally or parenterally) after which the bacterial vector vaccines express an engineered prokaryotic expression cassette contained therein that encodes a foreign antigen or antigens. Delivery of the foreign antigen to the host tissue using bacterial vector vaccines results in host immune responses against the foreign antigen.

Of the bacterial vector vaccines, live oral Salmonella vector vaccines have been studied most extensively. See U.S. Patent Publication No. 2007/0116725. Accordingly, in some embodiments, the live recombinant bacterial vector is a Salmonella vector. Bacterial vector vaccines are preferably genetically defined, attenuated, and well-tolerated by the subject, and retain immunogenicity. Hone et al., Vaccine, 9:810-816 (1991). A number of bacterial vector vaccines for the delivery of prokaryotic expression cassettes have been developed. Examples of bacterial that have been used as live recombinant bacterial vectors include Yersinia eternocolitica, Shigella spp., Vibrio cholera, Mycobactgerium strain BCG, and Listeria monocytogenes.

Treatment of Infection by Enterotoxigenic Escherichia coli

Another aspect of the invention provides a method of treating infection by enterotoxigenic Escherichia coli in a subject, by administering a therapeutically effective amount of antibody that specifically binds to an LngA epitope and a pharmaceutically acceptable carrier to the subject. Administering a therapeutically effective amount of an antibody that specifically binds to an LngA epitope provides temporary, passive immunity that can treat the infection. The present invention also provides antibodies capable of recognizing and specifically binding to LngA peptides, including antibodies derived from mixtures of such antigens or fragments thereof. An antibody “specifically binds” when the antibody preferentially binds a target structure, or subunit thereof, but binds to a substantially lesser degree or does not bind to a biological molecule that is not a target structure.

The antibodies of this invention are generated by conventional means utilizing the isolated, recombinant or modified antigens of this invention, or mixtures of such antigens or antigenic fragments. For example, polyclonal antibodies are generated by conventionally stimulating the immune system of a selected animal or human with the isolated antigen or mixture of antigenic proteins or peptides of this invention, allowing the immune system to produce natural antibodies thereto, and collecting these antibodies from the animal or human's blood or other biological fluid.

For example, an antibody according to the invention is produced by administering to a subject an LngA antigen or LngA composition of this invention. A suitable polyclonal antibody against epitopes of the LngA peptide may be generated as antisera. Furthermore, an antibody of the invention is isolated by affinity purifying antiserum generated during an infection of a mammal, e.g., a mouse, with enterotoxigenic Escherichia coli, using as immunoabsorbant the LngA peptide identified herein. Similarly, an antibody of the invention is isolated by immunizing mice with a purified, recombinant antigen of this invention, or a purified, isolated LngA protein of native origin.

Monoclonal antibodies (MAbs) directed against LngA peptide epitopes can also be generated. Hybridoma cell lines expressing desirable MAbs are generated by well-known conventional techniques, e.g. Kohler and Milstein and the many known modifications thereof. Similarly desirable high titer antibodies are generated by applying known recombinant techniques to the monoclonal or polyclonal antibodies developed to these antigens (see, e.g., Amit et al., Science, 233:747-753 (1986); Queen et al., Proc. Nat'l. Acad. Sci. USA, 86:10029-10033 (1989); and Riechmann et al., Nature, 332:323-327 (1988).

Given the disclosure contained herein, one of skill in the art may generate chimeric, humanized or fully human antibodies directed against the LngA peptide or antigenic fragments thereof by resort to known techniques by manipulating the complementarity determining regions of animal or human antibodies to the antigen of this invention. See, e.g., E. Mark and Padlin, “Humanization of Monoclonal Antibodies”, Chapter 4, The Handbook of Experimental Pharmacology, Vol. 113, The Pharmacology of Monoclonal Antibodies, Springer-Verlag (June, 1994).

### Vaccine and Therapeutic Compositions

The vaccine composition can include a pharmaceutically acceptable carrier, which constitutes one or more accessory ingredients. The term “pharmaceutically acceptable”, when used in reference to a carrier, is meant that the carrier, diluent or excipient must be compatible with the other ingredients of the formulation and not deleterious to the recipient thereof.

The vaccine composition may contain one or more of the isolated, recombinant, modified or multimeric forms of the LngA antigen of the invention, or mixtures thereof. Similarly, salts of the antigenic proteins may be employed in such compositions. A multivalent vaccine refers to a vaccine prepare using two or more different LngA antigens.

In another embodiment of this invention, a multivalent vaccine composition may include not only the LngA antigen or immunogenic fragment thereof, but may also include antigens from other disease-causing agents. Such other agents may be antigens from other Escherichia coli strains. Such other agents may be antigens from completely distinct bacterial pathogens or from viral pathogens. Combinations of the antigen(s) of this invention with other antigens or fragments thereof are also encompassed by this invention for the purpose of inducing a protective immune response in the vaccinated subject to more than a single pathogen. The selection of these other vaccine components is not a limitation of the present invention, and may be left to one of skill in the art.

Vaccines may be formulated for any of a variety of routes of administration as discussed further below. For example, vaccines may be formulated as a spray for intranasal inhalation, nose drops, swabs for tonsils, etc. Vaccines may be formulated for oral delivery in the form of capsules, tablets, gels, thin films, liquid suspensions and/or elixirs, etc. Optionally, the vaccine compositions may optionally contain adjuvants, preservatives, chemical stabilizers, as well as other conventionally employed vaccine additives. Typically, stabilizers, adjuvants, and preservatives are optimized to determine the best formulation for efficacy in the target human or animal. Suitable exemplary preservatives include chlorobutanol, potassium sorbate, sorbic acid, sulfur dioxide, propyl gallate, the parabens, ethyl vanillin, glycerin, phenol, and parachlorophenol.

As used herein, an “adjuvant” refers to any substance which, when administered with or before the LngA antigen, aids the LngA antigen in its mechanism of action. Thus, an adjuvant in a vaccine is a substance that aids the immunogenic composition in eliciting an immune response. One or more of the above described vaccine components may be admixed or adsorbed with a conventional adjuvant. Adjuvants may, in certain embodiments, enhance production of antibodies against enterotoxigenic Escherichia coli. Examples of suitable adjuvants include, but are not limited to, various oil formulations and/or emulsions such as stearyl tyrosine (see, for example, U.S. Pat. No. 4,258,029), muramyl dipeptide (also known as MDP, Ac-Mur-L-Ala-D), saponin, aluminum hydroxide, lymphatic cytokine, etc. Adjuvants that are particularly suitable for inducing mucosal immunity include, but are not limited to, Freund's adjuvant, cholera toxin (e.g., the Cholera toxin B subunit), heat labile enterotoxin (KT) from E. coli, Emulsomes (Pharoms, LTF., Rehovot, Israel), etc.

The invention thus also encompasses a prophylactic method entailing administering to an animal or human an effective amount of such a composition. The protein antigenic composition is administered in an “effective amount”, that is, an amount of antigen that is effective in a route of administration to provide a vaccinal benefit, i.e., protective immunity. Suitable amounts of the antigen can be determined by one of skill in the art based upon the level of immune response desired. In general, however, the vaccine composition contains between 1 ng to 1000 mg antigen, and more preferably, 0.05 μg to 1 mg per ml of antigen. Suitable doses of the vaccine composition of the invention can be readily determined by one of skill in the art. Generally, a suitable dose is between 0.1 to 5 ml of the vaccine composition. Further, depending upon the human patient or the animal species being treated, i.e. its weight, age, and general health, the dosage can also be determined readily by one of skill in the art.

In still another vaccine embodiment, the invention includes a composition which delivers passive protection against infection by the pathogen. For this composition, the antibodies against the LngA peptides disclosed herein are useful to provide to the subject a short-term, passive immune protection against infection. These passive immunity compositions may contain antibodies to other pathogens and suitable vaccine additives as described above, e.g., adjuvants, etc. These compositions may be administered in dosages similar to those described above for the compositions which actively induce immune protection in the vaccinated subject.

A variety of such pharmaceutically acceptable protein carriers, and/or components suitable for administration therewith may be selected by one of skill in the art. For example, pharmaceutical carriers include, without limitation, sterile saline, lactose, sucrose, calcium phosphate, gelatin, dextran,-agar, pectin, peanut oil, olive oil, sesame oil, and water. Additionally, the carrier or diluent may include a time delay material, such as glycerol monostearate or glycerol distearate alone or with a wax. In addition, slow release polymer formulations can be used. Liposomes or liposomal-like vehicles may also be employed.

Optionally, these compositions may also contain conventional pharmaceutical ingredients, such as preservatives, or chemical stabilizers. Suitable ingredients which may be used in a therapeutic composition in conjunction with the antibodies include, for example, casamino acids, sucrose, gelatin, phenol red, N-Z amine, monopotassium diphosphate, lactose, lactalbumin hydrolysate, and dried milk.

As a further embodiment of this invention, a therapeutic method involves treating a human or an animal for infection with enterotoxigenic Escherichia coli by administering an effective amount of such a therapeutic composition. An “effective amount” of a proteinaceous composition may be between about 0.05 to about 4000 μg/ml of an antibody or antigen of the invention. A suitable dosage may be about 1.0 ml of such an effective amount. Such a composition may be administered 1-3 times per day over a 1 day to 12 week period. However, suitable dosage adjustments for protein or nucleic acid containing compositions may be made by the attending physician or veterinarian depending upon the age, sex, weight and general health of the human or animal patient. Preferably, such a composition is administered parenterally, preferably intramuscularly or subcutaneously. However, it may also be formulated to be administered by any other suitable route, including orally or topically. The selection of the route of delivery and dosage of such therapeutic compositions is within the skill of the art.

In another embodiment, the LngA antigens, antibodies, and fragments of the invention, alone or in combination with other antigens, antibodies, and fragments from other microorganisms, may further be used in compositions directed to induce a protective immune response in a subject to the pathogen. These components of the present invention are also useful in methods for inducing a protective immune response in humans and/or animals against infection with enterotoxigenic Escherichia coli.

Alternatively, or in addition to the antigens or antibodies of the invention, other agents useful in treating the disease in question, e.g., antibiotics or immunostimulatory agents and cytokine regulation elements, are expected to be useful in reducing or eliminating disease symptoms. Such agents may operate in concert with the therapeutic compositions of this invention. The development of therapeutic compositions containing these agents is within the skill of one in the art in view of the teachings of this invention.

Antidiarrheal such as diphenoxylate, codeine phosphate, paregoric (camphorated opium tincture), loperamide hydrochloride, anticholinergics such as belladonna tincture, atropine, propantheline, kaolin, pectin, activated attapulgite, and the like.

### Administration

It will be appreciated that suitable routes of administration and dosing regimens may vary depending on various factors such as age, weight, height, sex, general medical condition, previous medical history, etc of the subject. Certain routes of administration (for example, intranasal and oral) and/or dosing regimens may be particularly suitable for use in humans.

In some embodiments of the invention, the composition is administered intranasally. Intranasal administration of the provided vaccine compositions may induce mucosal immune response and/or a serum antibody response. Intranasal administration may be advantageous over other routes of administration in certain aspects. For example, intranasal administration may obviate side effects such as nausea, stomach upset, or other digestive disturbance, that may accompany oral routes of administration. Compositions provided in the present disclosure may be administered intranasally, for example, via administration of a mist, aerosol, spray, and/or liquid droplets into the nose.

In some embodiments, compositions are designed such that exposure to the lungs is limited. For example, aerosols may be designed in such a way that the aerosol particle size limits the administration of the vaccine to the nasal pharynx, and not the oral pharynx or lungs. In such embodiments, large particles would stay in the nose, and few and/or very small particles would reach the alveoli in the lungs. The study of droplet size and point of delivery has been well studied for intranasal administration of medication. This expertise can be adapted to ensure that large droplets of the composition stay localized to the nasal mucosa. As another example, liquid droplets may be designed such that the volume of liquid delivered is below that which can flow or be aspirated down into the lungs.

In some embodiments of the invention, the composition is administered orally by ingestion. Vaccines may be formulated as, for example, capsules, tablets, gels, thin films, liquid suspensions and/or elixirs, etc.

Routes of administration also include subcutaneous, intradermal, and intramuscular. Routes of administration that may be particularly suitable for use in non-human animals include intragastric (via the stomach) and intraperitoneal. Intragastric administration may comprise, for example, oral gavage, which involves insertion of a tube containing the composition into the oesophagus and delivering the composition directly into the stomach using a syringe or pump. In some embodiments of the invention, the composition is administered intraperitoneally, across the peritoneum. Intraperitoneal administration may comprise, for example, injection of the composition into the peritoneal space using a syringe applied to the abdomen.

Dosing regimens may comprise a single immunization or multiple immunizations. For example, vaccines may be given as a primary immunization followed by one or more boosters. Boosters may be delivered via the same and/or different route as the primary immunization. Boosters are generally administered after a time period after the primary immunization or the previously administered booster. For example a booster can be given about two weeks or more after a primary immunization, and/or a second booster can be given about two weeks or more after the first boosters. Boosters may be given repeatedly at time periods, for example, about two weeks or greater throughout up through the entirety of a subject's life. Boosters may be spaced, for example, about two weeks, about three weeks, about four weeks, about one month, about two months, about three months, about four months, about five months, about six months, about seven months, about eight months, about nine months, about ten months, about eleven months, about one year, about one and a half years, about two years, about two and a half years, about three years, about three and a half years, about four years, about four and a half years, about five years, or more after a primary immunization or after a previous booster.

The following example is included for purposes of illustration and is not intended to limit the scope of the invention.

### EXAMPLE

### Murine Immunization with CS21 Pili or LngA Major Subunit of Enterotoxigenic Escherichia coli (ETEC) Elicits Systemic and Mucosal Immune Responses and Inhibits ETEC Gut Colonization

CS21 pili of enterotoxigenic Escherichia coli (ETEC) is one of the most prevalent ETEC colonization factors. CS21 major subunit, LngA, mediates ETEC adherence to intestinal cells, and contributes to ETEC pathogenesis in a neonatal mouse infection model. The objectives of this work were to evaluate LngA major subunit purified protein and CS21 purified pili on immunogenicity and protection against ETEC colonization of mice intestine. Recombinant LngA purified protein or purified CS21 pili from E9034A ETEC strain were evaluated for immunogenicity after immunization of C57BL/6 mice. Specific anti-LngA antibodies were detected from mice serum, feces, and intestine fluid samples by ELISA assays. Protection against gut colonization was evaluated on immunized mice orally challenged with wild type E9034A ETEC strain and by subsequent quantification of bacterial colony forming units (CFU) recovered from feces. Recombinant LngA peptide and CS21 pili induced specific humoral and mucosal anti-LngA antibodies in the mouse model. CS21 combined with CT delivered intranasally as well as LngA combined with incomplete Freund adjuvant delivered intraperitoneally inhibited ETEC gut colonization in a mouse model. In conclusion, both LngA purified protein and CS21 pili from ETEC are highly immunogenic and may inhibit ETEC intestinal shedding. Our data on immunogenicity and immunoprotection indicates that CS21 is a suitable vaccine candidate for a future multivalent vaccine against ETEC diarrhea.

### Materials and Methods

Bacterial Strains

Bacterial strains used in this study are described in Table 1. E. coli DH5a (Life Technologies, Grand Island, N.Y.), and CS21⁺ or CS8⁺ ETEC strains were cultured on Luria broth (LB), Terrific broth (TB) or TB agar plates at 37° C. overnight. Clavijo et al., Microb. Pathog. 48(6):230-38 (2010). CS21⁺ ETEC E0934A strain was electroporated with plasmid pCM17 containing luciferase genes. Rhee et al., Gut Microbes. 2(1):34-41 (2011). The pCM17 was kindly provided by Dr. James B. Kaper. E9034A/pCM17, used for in vivo challenge mice experiments, was cultured in LB broth supplemented with Kanamycin at 37° C. overnight and then sub-cultured in DMEM/F12 (1:1) medium (25 mM glucose, 15 mM HEPES and 0.5% mannose) at ratio of 1:10 for 3 hours at 37° C. The bacteria were washed once with sterile PBS and the bacterial concentration was adjusted in sterile PBS before challenge.

TABLE 1
Strains and their characteristics in this study.

| | | | | LngA | Phylogenetic | |
|---|---|---|---|---|---|---|
| Strains | Serotype | Toxin | Pili | alleles | groups | Countries |
| DH5α | N.K. | None | None | N.A. | N.K. | N.K. |
| E9034A/ | O8:H9 | LT, ST | CS21, CS3 | Group 1 | A | N.A. |
| pCM17 | | | | | | |
| E9034A | O8:H9 | LT, ST | CS21, CS3 | Group 1 | A | Caribbean |
| ΔlngA | | | | | | |
| E9034A | O8:H9 | LT, ST | CS21, CS3 | Group 1 | A | Caribbean |
| 10159-a | N.K. | LT | CS21, | Group 2 | B1 | Chile |
| COCt129 | O128:H45 | ST | CS21, CFA/I | N.K. | N.K. | Colombia |
| COCt40 | O25:H16 | LT | CS21, CS6 | N.K. | N.K. | Colombia |
| M452-C1 | O20:H- | ST | CS21, CFA/I | Group 2 | A | Morocco |
| B2C | O6:H16 | LT, ST | CS21, CS2, | Group 1 | A | Vietnam |
| | | | CS3 | | | |
| B7A | O148:H28 | LT, ST | CS21, CS6 | Group 2 | B1 | Vietnam |
| M403-C3 | N.K. | N.K. | CS8 | N.A. | A | Bangladesh |
| E2528C1 | O25:NM | LT | CS8, CS14 | N.A. | A | Caribbean |
| WS6866B-2 | N.K. | LT | CS8 | N.A. | N.K. | Egypt |

CS = coli surface antigen,
CFA/I = colonization factor antigen I,
NM = nonmotile,
N.A. = not applicable,
N.K. = not known,
LT = heat-labile enterotoxin,
ST = heat-stable enterotoxin.
LngA and CS21 isolation and purification

The recombinant LngA-His protein was prepared by GenScript (Piscataway, N.J.). Briefly, the LngA gene derived from wild-type ETEC E9034A along with the 6×His tag (SEQ ID NO: 7) DNA sequence were synthesized and subcloned into pET15b vector. E. coli BL21 (DE3) was transformed by the recombinant plasmid and induced by IPTG to overexpress LngA-His recombinant protein. Bacteria culture was harvested by centrifugation, lysed by sonication, and the lysate supernatant was subject to purification by a Ni-NTA column. CS21 or CS8 pili were purified as described before from wild-type ETECs. Girón et al., Mol. Microbiol. 12(1):71-82 (1994). Briefly, wild-type ETECs were cultured on TB agar plates overnight at 37° C., harvested in sterile phosphate buffer solution (PBS), vortexed for one minute, and centrifuged at 8,000×g and 4° C. for 20 minutes. The supernatant was centrifuged at 12,000×g and 4° C. for 30 minutes and subsequently centrifuged at 40,000×g at 4° C. for 30 minutes. The pellet containing CS21 or CS8 pili was resuspended in sterile PBS and stored at −80° C. until use. The protein concentration was determined by Pierce BCA Protein Assay Kit (Thermo Fisher Scientific, Waltham, Mass.) according to manufacturer's instructions.

Multiple Protein Sequence Alignment and B Cell Epitope Prediction

A multiple protein sequence alignment was generated by the DNADynamo™ software by using LngA amino acid sequences derived from CS21⁺ ETEC strains E9034A (NG_036490.1), M526-C6B (EU107107.1), BR5 (EU107105.1), MP215-1 (EU107104.1), M633-C1 (EU107103.1), M626-C(EU107102.1), M452-C1 (EU107101.1), M445-C1 (EU107100.1), G1026 (EU107099.1), B7A(EU107098.1), 2108-2 (EU107097.1), 11381a (EU107096.1), 10159a (EU107095.1), M408 (EU107094.1), P307 (EU107093.1), M424-C1 (EU107092.1), M145-C2 (EU107091.1), B2C (EU107090.1), 01117-5 (EU107089.1). The GeneBank accession numbers for CofA, BfpA, and TcpA proteins are CEJ09700.1, WP_000253758.1, and AAL58332.1, respectively. The linear B cell epitopes of LngA major subunit derived from strain E9034A were predicted by Bepipred Linear Epitope Prediction (Larsen et al., Immunome Res. 2:2 (2006)), Parker Hydrophilicity Prediction (Parker et al., Biochemistry (Mosc.). 25(19):5425-32 (1986)), and Emini Surface Accessibility Prediction (Emini et al., J. Virol. 55(3):836-39 (1985)) of Antibody Epitope Prediction on IEDB Analysis Resources.

Immunogenicity Experiments

All animal experiments performed in this study were approved by the IACUC at Vanderbilt University under protocol M/11/223. For LngA immunogenicity studies, twenty-five 7- to 8-weeks old C57BL/6 mice were purchased from Jackson Laboratories and divided into five groups with five mice per group. Mice in each group received by intraperitoneal injection a combination of one antigen or placebo plus one adjuvant (1:1, v/v): group A: PBS plus incomplete Freund's adjuvant (IFA) (Sigma, St. Louis, Mo.); group B: 30 μg LngA plus IFA; group C: PBS plus aluminum hydroxide (ALUM), (InvivoGen, San Diego, Calif.); group D: 30 μg LngA and Alum; group E: 30 μg LngA and 2 μg cholera toxin (CT) per mouse. Mice were immunized three times by intraperitoneal injection at two-week intervals. Blood and fecal samples were collected one day before each immunization and 10 days after the final immunization by submandibular vein or natural defecation, respectively. Serum samples were obtained after blood samples were clotted and centrifuged. Fecal samples were weighed, suspended in reconstitution buffer (10 mM Tris, 100 mM NaCl, 0.05% Tween-20, 5 mM sodium azide, pH 7.4) supplemented with 1× protease inhibitor cocktail (Thermo Fisher Scientific, Waltham, Mass.) at 5:1 ratio (5 ml reconstitution buffer per gram of feces) and centrifuged. The clear fecal antibody suspension in the supernatant was collected and stored at −20° C. for further studies. In addition to serum and stools, intestinal fluid samples were also collected after the mice were euthanized. One gram of intestinal fluid was homogenized with 2.5 mLs of reconstitution buffer with 1× protease inhibitor cocktail and centrifuged. The cleared intestinal fluid in the supernatant was collected and stored at −20° C. until used.

Immunoprotection Experiments

Mice were immunized with either LngA-His recombinant protein or purified CS21 pili as described above and then challenged with the wild-type CS21-expressing ETEC E9034A strain to evaluate for protection against intestinal colonization by ETEC. Two independent experiments were conducted, one tested LngA purified protein and another tested native CS21 purified pili. In the LngA immunoprotection study, 7- to 8-week old C57BL/6 mice were divided into four groups with five mice per group. Each group received different combinations of vaccine or placebo plus adjuvants by intraperitoneal injection: group A: no vaccine (naïve group); group B: PBS and IFA (1:1, v/v); group C: 30 μg LngA and IFA (1:1, v/v); group D: 30 μg LngA and 2 μg CT per mouse.

For CS21 immunoprotection study, forty-five 7- to 8-week old C57BL/6 mice were divided into nine groups with five mice per group. Each group received different combinations of vaccine or placebo plus adjuvants by subcutaneous (SC), intranasal (IN), or intraperitoneal (IP) route: group A: PBS and ALUM (1:1, v/v) in 200 μl volume by the SC route; group B: 50 μg CS21 and ALUM (1:1, v/v) in 200 μl volume by the SC route; group C: PBS and 2 μg CT in 20 μl volume by the IN route; group D: 20 μg CS21 and CT in 20 μl volume by the IN route; group E: PBS and IFA (1:1, v/v) in 300 μl volume by the IP route; group F: 50 μg CS21 and IFA (1:1, v/v) in 300 μl volume by the IP route; group G: PBS and ALUM (1:1, v/v) in 300 μl volume by the IP route; group H: 50 μg CS21 and ALUM (1:1, v/v) in 300 μl volume by the IP route; group I: no vaccine (naïve group). For antibody titration, blood and fecal samples were collected before the primary immunization and seven days after the final immunization and the intestine wash samples were collected after euthanasia as described above.

Eight days after final immunization, each group of mice were given streptomycin (5 g/L) for 24 hours and were switched to regular water for 24 hours. The purpose of the streptomycin was to depopulate mice intestine normal flora and facilitate the ETEC challenge strain colonization of the mouse intestine. Rhee et al., Gut Microbes. 2(1):34-41 (2011). Ten days after the final immunization, all groups except the naïve group (no vaccine group) were challenge by oral gavage with 1×10⁸ or 1×10⁶ CFU wild-type ETEC strain E9034A carrying luciferase gene plasmid pCM17 for LngA or CS21 immunoprotection study, respectively. In vivo bioluminescence imaging was carried out by in vivo imaging system XENOGEN IVIS 200 (Xenogen Corporation, Alameda, Calif.) before challenge and daily after challenge to quantify the number of colonizing bioluminescent ETEC.

In addition to measuring bioluminescense to evaluate the ETEC colonization level, ETEC colony forming units (CFU) were also calculated from fecal samples collected daily from each mice after challenge. Bacterial shedding is used as a murine marker of intestinal colonization. Accordingly, feces were collected, weighed, suspended and diluted in sterile PBS, and plated on MacConkey agar to determine the E. coli CFU/g feces.

ELISA Assays

Indirect ELISA assays were used for titration of anti-LngA and anti-CT antibodies from serum, fecal, and intestinal samples of immunized mice. CS21 pili were coated on ELISA plates to titrate anti-LngA antibody-containing sera from mice immunized with LngA-His recombinant protein. Similarly, LngA-His protein was coated on ELISA plates to titrate anti-CS21 antibody-containing sera from mice immunized with CS21. These coating strategies would minimized the recognition of antibodies not directed to CS21 or to LngA, including anti-His antibodies. Briefly, Immulon 2 HB 96 well microtiter plates (Thermo Fisher Scientific, Waltham, Mass.) were coated in duplicates with 25 ng of purified CS21 or LngA, 500 ng of purified CS21 or LngA, or 500 ng of purified CT (Sigma-Aldrich, St. Louis, Mo.) in 100 μl Antigen Coating Buffer (15 mM Na₂CO₃, 35 mM NaHCO₃, pH 9.6) and incubated at 37° C. for one hour. Plates were washed three times with 200 μl PBS supplemented with 0.05% Tween-20 (PBS-T) per well and blocked with 10% skim milk/PBS-T at 37° C. for one hour and washed three times with 200 μl PBS-T per well. Serum, fecal, and intestinal wash antibody samples were serially diluted in PBS-T (fecal samples) or 5% skim milk/PBS-T (serum and intestine wash samples) and incubated at 37° C. for one hour. Plates were washed three times with 200 μl PBS-T per well. The horseradish peroxidase (HRP) conjugated goat anti-mouse IgG (1:5000) or IgA (1:1000) diluted in PBS-T was added to each well and plates were incubated at 37° C. for one hour. Plates were washed three times with 200 μl PBS-T per well. One hundred microliters 3,3′,5,5′-Tetramethylbenzidine (TMB) substrate (KPL, Gaithersburg, Md.) were added to each well to incubate at room temperature for 20 minutes and 100 μl stop solution were added to stop the reaction. The optical density (OD) value was measured at 450 nm. The antibody titers were presented as the log 10 value of the antibody dilution when OD_{450 nm} was 0.4. Samples with OD_{450 nm} less than 0.4 are considered as negative.

Polyacrylamide Gel Electrophoresis and Immunoblotting

LngA-His recombinant protein was prepared by GenScript (Piscataway, N.J.) and CS21 and CS8 pili were purified from ETEC strains of different countries as described above. SDS-polyacrylamide gel electrophoresis (SDS-PAGE) gels were stained with Coomassie blue and processed for immunoblotting with anti-LngA monoclonal antibody or mice antisera from this immunization study. Pili preparations of laboratory reference strain DH5a and CS21⁺ ETEC strain E9034A were used as negative and positive controls and LngA-His recombinant protein was used as purified protein control. Pili and LngA-His recombinant proteins were separated by electrophoresis on pre-casted 4-20% SDS-PAGE gel (Bio-Rad, Hercules, Calif.) and were either stained with Bio-Safe Coomassie G-250 stain (Bio-Rad, Hercules, Calif.) for one hour and then washed with water or transferred to nitrocellulose membrane by Semi-dry Blotting Unit Electrophoresis System (Thermo Fisher Scientific, Waltham, Mass.). The nitrocellulose membrane was blocked with 10% skim milk/PBS-T for one hour, washed three times with PBS-T, probed with anti-LngA monoclonal antibody or mice antisera (derived from CS21 and LngA immunization study) in 2% skim milk/PBS-T for one hour, washed three times with PBS-T, probed by IRDye® 680LT goat anti-mouse IgG (LI-COR, Lincoln, Nebr.) for one hour, washed three times with PBS-T, and scanned by the LI-COR Odyssey Infrared Imaging System at 700 nm channel.

Statistical Analysis

Statistical analysis of data were conducted by GraphPad Prism 6 (San Diego, Calif.) using Fisher test. P-values less than 0.05 were considered statistically significant.

### Results

Variable Regions within the LngA Peptide Overlap with Surface Accessible B Cell Epitopes

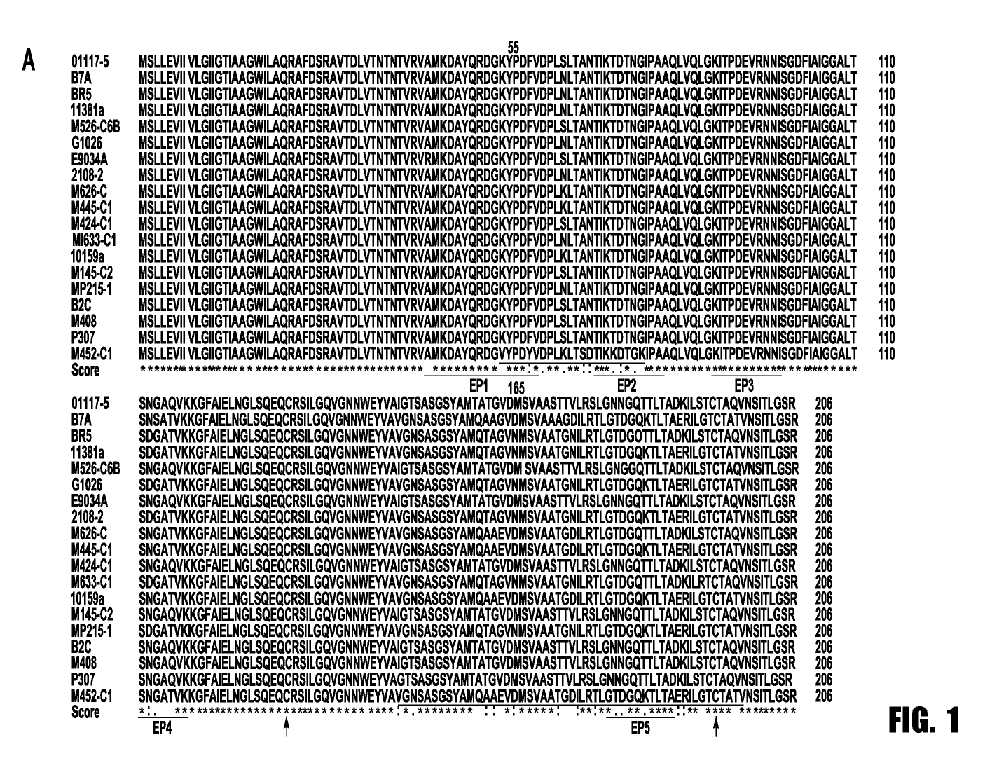

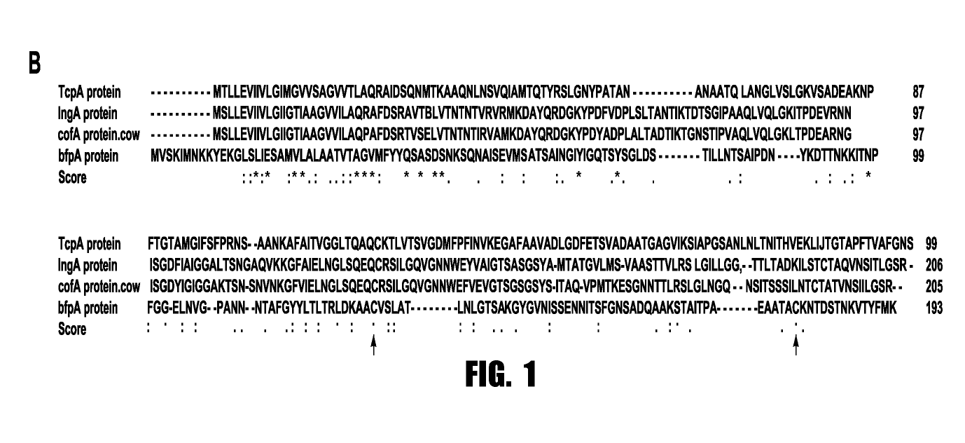

Two major variable regions were identified within the LngA peptide sequence when 19 CS21⁺ ETEC strains LngA sequences were aligned and compared (FIG. 1A). The variable regions spanned from residues 56 to 77 and 148 to 197. Cysteines at LngA residues 132 and 194 were conserved among all LngA variants (FIG. 1A). Equivalent disulfide-bond-forming cysteines were identified among homologous pili major subunits including CofA (ETEC CS8) residues 131 and 193, BfpA (EPEC) residues 129 and 179, and TcpA (Vibrio cholerae) residues 120 and 186 (FIG. 1B). Linear B cell epitopes (FIG. 2A), hydrophilicity regions (FIG. 2B), and surface accessibility sequences (FIG. 2C) were identified within the LngA sequence Five potential surface accessible and hydrophilic B cell epitopes were identified at residues ⁵⁰AYQRDGKYPDFV⁶¹ (SEQ ID NO: 1), ⁷⁰TIKTDTSGIP⁷⁹ (SEQ ID NO: 2), ⁸⁹ITPDEVRNN⁹⁷ (SEQ ID NO: 3), ¹⁰⁹LTSNGAQVK¹¹⁷ (SEQ ID NO: 4), ¹⁷⁸GNNGQTTLT¹⁸⁶ (SEQ ID NO: 5), and tentatively designated as epitopes 1 to 5 (EP1 to 5), respectively (FIGS. 1A and 2). Predicted epitopes 1, 2 and 5 contained 2, 4 and 3 single amino acid polymorphisms, respectively (FIG. 1A).

LngA-His recombinant and CS21 protein preparations stimulated murine anti-LngA antibodies cross-reacts with CS21 and CS8 homologues.

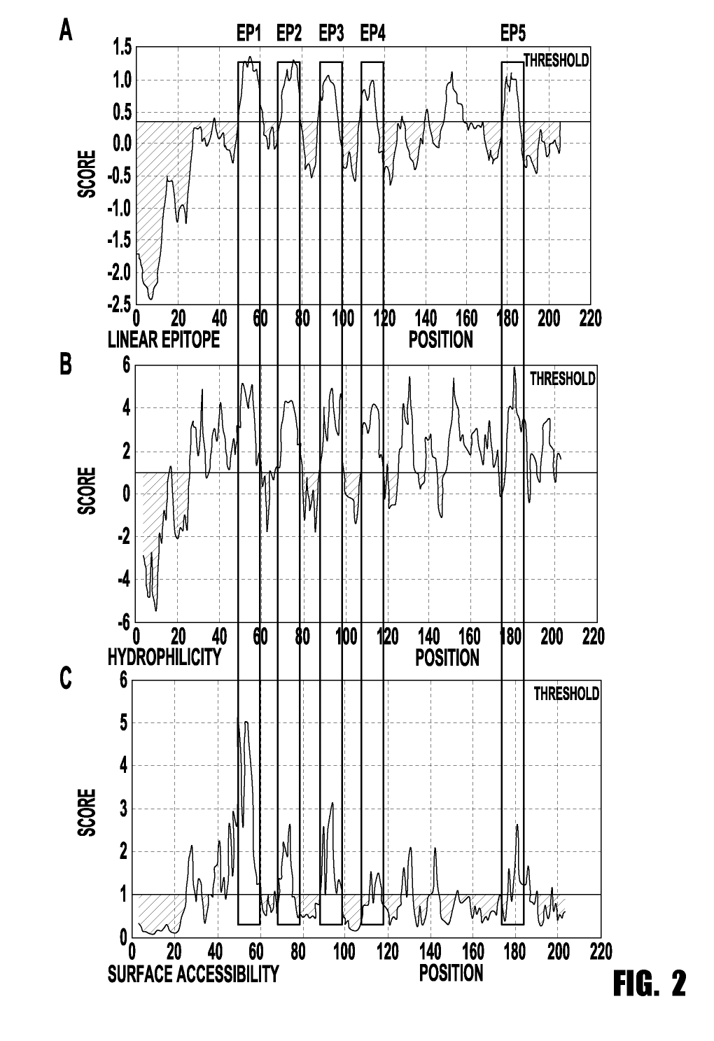

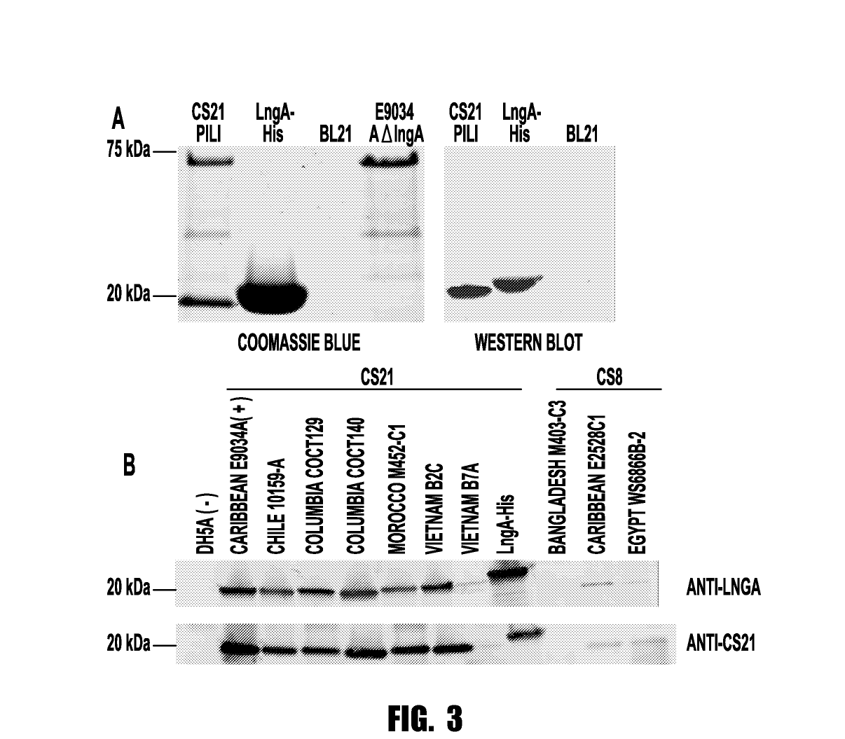

LngA-His recombinant protein and CS21 native pili derived from E904A ETEC strain were analyzed by SDS-PAGE to confirm protein purity and stability before immunization experiments. LngA-His recombinant and CS21-derived native LngA peptides were visualized in the Coomassie stained gel and they were specifically recognized in Western blot by anti-LngA monoclonal antibody (FIG. 3A). Coomassie stained gels of denatured CS21 purified pili showed not only the LngA pilin (22 kDa) subunit but also an unknown 70 kDa protein. Based on band size and intensity, both proteins were present at similar concentrations (FIG. 3A). Although the 70 kDa unknown protein was not recognized by the anti-LngA monoclonal antibody, it is unique to ETEC, since it was also produced by the E9034AΔlngA mutant strain and it was not produced by the E. coli BL21 strain (FIG. 3A).

To test for anti-LngA antibody cross-reactivity with CS21 or CS8 major subunits, worldwide ETEC strains expressing CS21 or CS8 major subunit alleles were tested by immunoblot using mice serum raised against LngA and CS21 protein derived from CS21⁺ ETEC E9034A strain (Table 1). Pili preparation of laboratory reference E. coli DH5a and CS21⁺ ETEC E9034A strains were used as negative and positive controls for LngA immunoblotting, respectively. LngA-His recombinant protein was used as protein positive control. The proteins were probed by anti-LngA mice sera. As shown in FIG. 3B, anti-LngA antibody raised against LngA-His and CS21 pili derived from E9034A had strong reaction with LngA derived from CS21⁺ ETEC strains E9034A (Caribbean), 10159-a (Chile), COCt129 (Colombia), COCt40 (Colombia), M452-C1 (Morocco), and B2C (Vietnam) and weak reaction to LngA of B7A (Vietnam). Weak signal was also observed for the CofA major subunit of CS8⁺ ETEC strains E2528C1 (Caribbean) and WS6866B-2 (Egypt). The only protein no recognized by anti-LngA sera was CofA from ETEC M403-C3 ETEC (Bangladesh) strain.

Both LngA and Purified CS21 Pili Elicit Strong Anti-LngA Antibody Responses

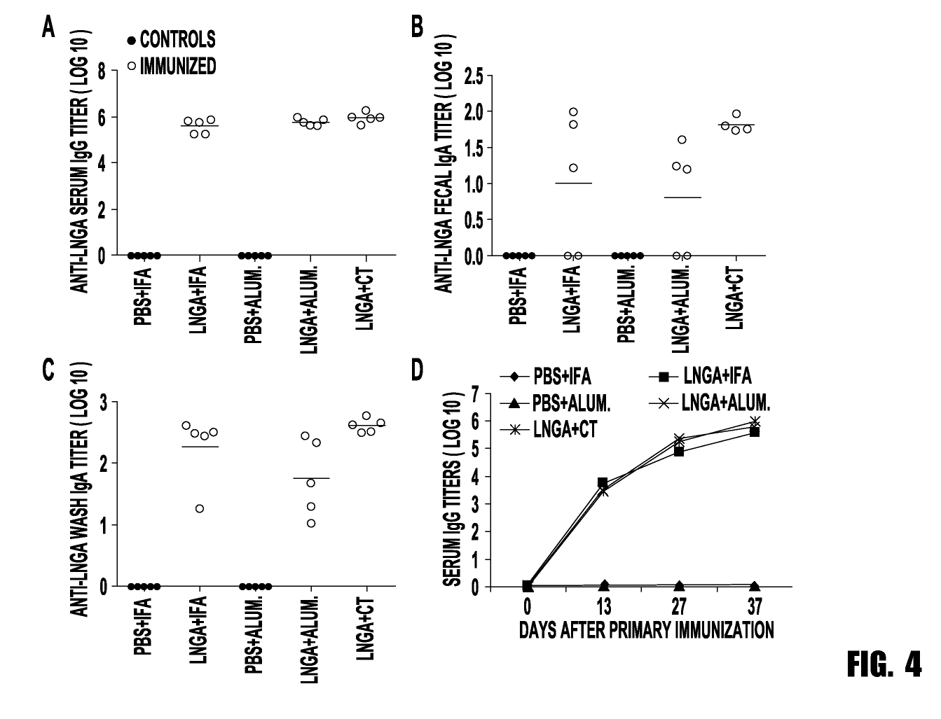

To evaluate CS21 pili and LngA major subunit immunogenicity, we immunized C57BL/6 mice with purified CS21 pili or LngA-His recombinant protein by subcutaneous (SC), intranasal (IN), or intraperitoneal (IP) injection and titrated specific anti-LngA antibodies in serum, fecal, and intestinal wash samples by ELISA. Mice immunized by IP route with LngA plus incomplete IFA, ALUM, and CT mounted significant anti-LngA serum IgG responses (titers (Log 10)=5.6, p<0.01; 5.8, p<0.01; 6, p<0.01, respectively) compared with mock immunized controls (FIG. 4A). The serum IgG responses from mice immunized with LngA plus three different adjuvants were seen as early as 13 days after the primary immunization and increased steadily overtime until reaching a peak response at 37 days after primary immunization (FIG. 4D). In addition, mice immunized with LngA plus IFA, ALUM, and CT mounted significant anti-LngA fecal IgA (titers (Log 10)=1.0, p=0.05; 0.8, p=0.04; 1.8, p<0.01, respectively) (FIG. 4B) and intestinal wash IgA responses (titers (Log 10)=2.3, p<0.01; 1.8, p<0.01; 2.6, p<0.01, respectively) (FIG. 4C).

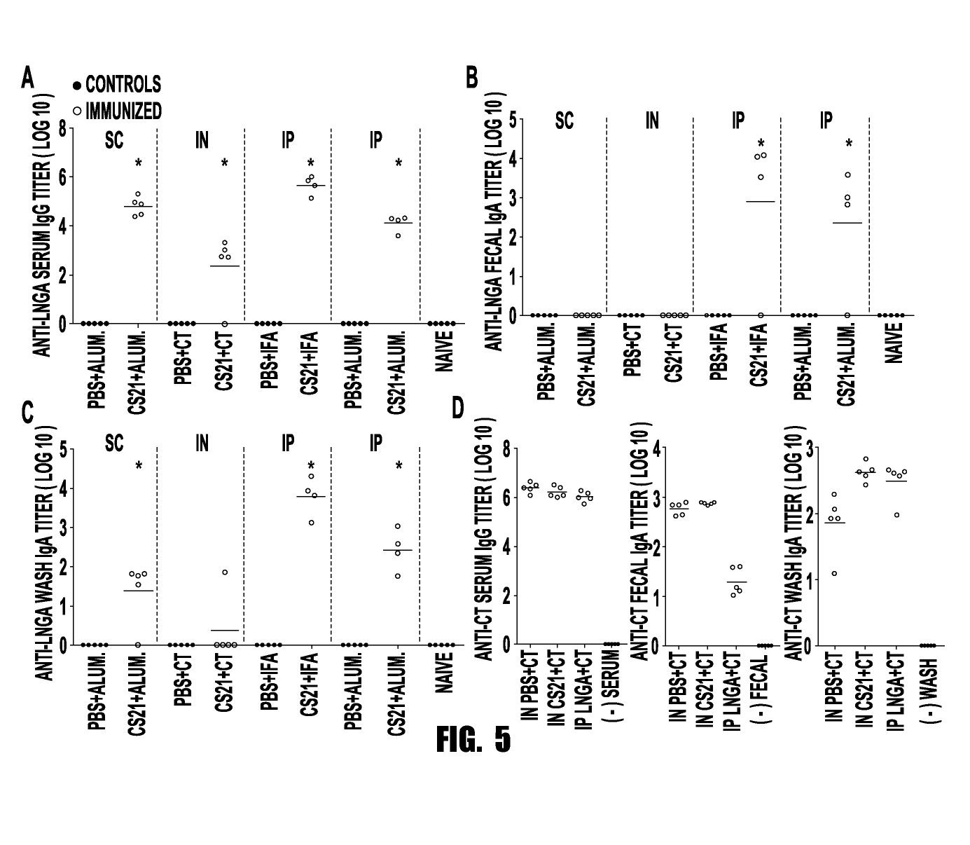

Mice immunized with CS21 plus ALUM by SC route, CS21 plus CT by IN route, CS21 plus IFA by IP route, and CS21 plus ALUM by IP route mounted significant anti-LngA serum IgG responses (titers (Log 10)=4.8, p<0.01; 2.4, p<0.01; 5.7, p<0.01; 4.1, p<0.01 respectively) compared with mock immunized controls (Figure SA). In addition, mice immunized with CS21 mounted significant anti-LngA fecal IgA (titer (Log 10) of CS21 plus IFA by IP=2.9, p=0.0116; titer (Log 10) of CS21 plus ALUM by IP=2.4, p=0.0123, respectively) (FIG. 5B) and intestinal wash IgA responses (titers (Log 10)=1.4, p<0.01; 0.4, p=0.3466; 3.8, p<0.01; 2.4, p<0.01 respectively) (FIG. 5C).

Since CT is not only a potent adjuvant but an immunogen, when delivered as adjuvant with CS21 or LngA, it stimulated strong serum anti-CT IgG, fecal IgA, and intestinal wash IgA antibody responses (FIG. 5D).

LngA and CS21 immunization inhibits wild-type ETEC gut colonization in mice

To evaluate the role of CS21 and LngA in immunoprotection against mice gut colonization, mice immunized with LngA peptide or with CS21 pili were orally challenged with CS21⁺ wild type ETEC E9034A strain and subsequently evaluated for gut colonization by quantifying bacteria shedding from stools.

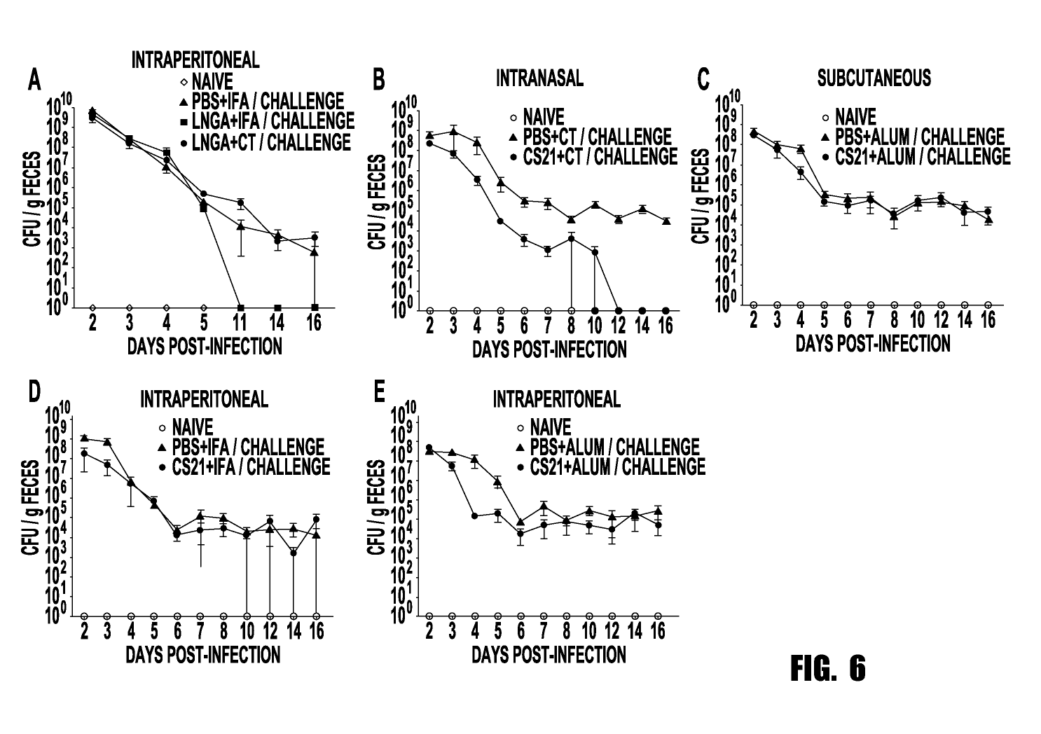

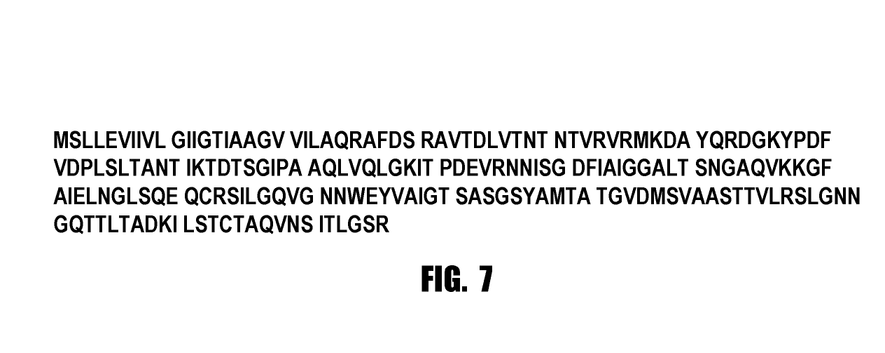

For mice immunized IP with LngA, the LngA+IFA immunized group shed less bacteria in feces 5 days after challenge than the negative control group or the LngA+CT group (FIG. 6A). LngA+IFA immunized group stopped shedding bacteria in the stools from day 11 after challenge. For mice immunized IN with CS21, the CS21+CT immunized group shed significantly less bacteria in feces than the unimmunized control or the remaining immunized groups (FIG. 6B). The CS21+CT IN immunized group stopped shedding bacteria in the stools from day 12 after challenge. In contrast, all remaining CS21 immunized groups and controls continue shedding bacteria in the stool after day 16 (FIG. 6 panels C-E). Differences in bacterial shedding in mice immunized SC or IP with CS21+IFA or CS21+ALUM. were only significant at certain days after challenge (FIG. 6 panels C-E). In summary, IP LngA+IFA and IN CS21+CT vaccine strategies resulted in lower fecal bacterial shedding after ETEC oral challenge of C57BL/6 mice.

### Discussion

ETEC isolates reaching the gastrointestinal tract via oral transmission, bind to intestinal epithelial cells through CSs, and colonize the intestinal mucosal surface. CSs-based vaccines may elicit specific immune responses that by blocking CSs-mediated ETEC adherence to intestinal cells they may inhibit ETEC gut colonization, and prevent diarrhea. Unlike other CSs such as CFA/I, CS1, CS5, and CS8 which have been intensively studied as potential vaccine candidates, the immunogenicity and immunoprotective activity of CS21 and its major subunit LngA have not been explored. This study showed that native CS21 and LngA recombinant proteins are highly immunogenic in the murine model and they elicit a robust systemic IgG and mucosal secretory IgA responses. Under unique delivery and adjuvant conditions, LngA and CS21 inhibited ETEC gut colonization measured by the lower fecal shedding among immunized mice compared to controls. This suggested that anti-CS21 and anti-LngA immune responses may inhibit CS21-mediated ETEC binding to intestinal cells and may block gut colonization. Evidence that anti-LngA antibodies inhibited CS21-dependent ETEC adherence to intestinal cells was previously demonstrated in vitro. Guevara et al., Microbiol. Read. Engl. 159(Pt 8):1725-35 (2013).

ETEC CSs have been tested as vaccines to protect against ETEC colonization and diarrheal diseases in various forms including live attenuated bacteria, inactivated bacteria, and protein subunits. In one vaccine study in animals, attenuated ETEC strains expressing the CFA/I and a detoxified heat-labile enterotoxin (LThK63) enhanced clearance of ETEC from the lungs of mice and protected mice from intestinal ETEC colonization and LT-induced fluid accumulation. Byrd W, Boedeker EC., Vet. Immunol. Immunopathol. 152(1-2):57-67 (2013). In one human volunteer study, a three-strain-combination live attenuated vaccine known as ACE527 was created to include strain ACAM2025 expressing CFA/I and LTB; strain ACAM2022 expressing CS5, CS6, and LTB; and strain ACAM2027 expressing CS1, CS2, CS3, and LTB. This vaccine was well tolerated and at least 50% of subjects in the high-dose group responded to LTB, CFA/I, CS3, and CS6. Harro et al., Clin. Vaccine Immunol. CVI. 18(12):2118-27 (2011). In addition, it reduced the incidence and severity of diarrhea in a human challenge model of diarrheal disease. Darsley et al., Clin. Vaccine Immunol. CVI. 19(12):1921-31 (2012). In preclinical vaccine development studies, oral inactivated ETEC prototype vaccines producing CFA/I or CFA/I+CS3+CS5+CS6 and recombinant LTB/CTB hybrid protein were shown to be safe and well tolerated and stimulated significant systemic and mucosal immune responses to each of the vaccine CF antigens as well as to LT B subunit. Holmgren et al. Vaccine, 31(20):2457-64 (2013). In one ETEC subunit vaccine study, epitopes from seven CS were genetically fused to STa-LT toxoid constructs to create multi-epitope fusion antigens (MEFAs) CFA/I/II/IV-STaA14Q-dmLT and CFA/I/II/IV-STaN12S-dmLT. Antibodies induced by the MEFAs showed in vitro adherence inhibition activities against ETEC or E. coli strains expressing these seven CFAs and neutralization activities against both toxins. Ruan et al., Clin. Vaccine Immunol. CVI. 21(2):243-49 (2014). Based on our immunoprotection data and previously published studies reporting high global prevalence of CS21 among ETEC strains, the inventors believe that CS21 should be included as an antigen in new multivalent ETEC vaccines to tested for protection against toxin-mediated diarrhea in the human host.

Antigenic variations were observed at the predicted LngA surface accessible B-cell epitopes. While LngA antigenic variation may be a critical mechanism to evade host memory immune responses and facilitate the re-infection (Kotloff et al., The Lancet. 382(9888):209-22 (2013)), the number of B-cell epitopes was high enough to allow for antigen recognition among different LngA variants. Anti-LngA antibodies have strong cross-reactivity with genetically diverse LngA peptides and weak cross-reactivity with evolutionally related CS8 pili, as demonstrated by immunoblotting analysis. This indicates that anti-LngA antibody has the potential to provide broad protection against ETECs expressing type 4 pili cross-reactive variants.

Anti-LngA antibodies are not the only explanation for the protection against gut colonization observed with LngA+IFA vaccine given IP or the CS21+CT vaccine given IN. Interestingly, all antigen-adjuvant combinations delivered by any route were successful in eliciting strong and specific anti-LngA or anti-CS21 antibody responses yet only two vaccine combinations—LngA+IFA given IP or the CS21+CTgiven IN—were associated with protection. Since no protection against colonization was observed with LngA+CT given IP, or with CS21+ALUM or CS21+IFA given IP or SC, we believe that specific anti-LngA or anti-CS21 non-antibody immune responses should contribute to protection against colonization. Further experiments are necessary to elucidate the specific factors involved in protection when using LngA or CS21 as immunogens. Comparing LngA and CS21 head to head or in combination with different adjuvants and by different delivery routes may provide critical information on how to optimize LngA or CS21 immunoprotection.

Overall, IN delivery of CS21 pili in combination with CT outperformed recombinant IP delivery of LngA plus IFA in protecting mice against ETEC intestinal colonization based on reduced fecal ETEC shedding. One explanation for better protection with CS21 compared with LngA is that recombinant LngA peptide may have altered tertiary structures with respect to the native CS21 LngA conformation. As a consequence, anti-LngA antibodies derived from recombinant LngA may react less efficiently against the native LngA peptide in CS21 compared with those derived from purified CS21 pili. An additional explanation for better protection with CS21 pili may has to do with CS21+ETEC co-purified proteins. A 70 kDa protein (FIG. 5A) and other subunits of CS21 pili, e.g. minor pilin subunit LngB, may contribute additional protection compared to LngA alone against ETEC challenge and colonization. The role of the unknown 70 kDa protein and LngB in stimulation of protective immune responses is yet to be defined. It is possible that additional ETEC surface protein traces co-precipitated in the CS21 preparation may also contribute to immunoprotection. CS3, among them is expressed by E9034A ETEC strain (Levine et al., Infect. Immun 44(2):409-20 (1984)) and it is also associated with cell adherence to intestinal epithelial cells.

LngA- and CS21 immunized mice were challenged with bioluminescent ETEC and the intestinal bacterial load was evaluated by measuring bioluminescence. During the first three days of daily measurements there were no significant differences between control and immunized groups (data not shown) and days later the bioluminescence signal was very low or undetectable. Limited bioluminescence detection may explain the low sensitivity of detection in this colonization model. Also, the high challenge dose of 1×10⁸ CFU per mice, may have overwhelmed mucosal immune responses and lead to high ETEC colonization in both control and immunized groups with no appreciable differences. In contrast, CFU detected in feces was a more sensitive method of measuring ETEC intestinal colonization as CFU shedding remained positive one week after bioluminescence signal became negative. By measuring CFU it was shown that ETEC were cleared in LngA-immunized mice 11 days post challenge, suggesting that immunized mice are more prone to clear ETEC colonization from mice intestines after intestinal ETEC load decreases over time. One limitation of this quantitative assay is that ETEC stool shedding may not accurately indicate the level of ETEC colonization in the small or large intestines. Future in vivo experiments may need to address the level of ETEC gut colonization after oral infections and the protection against gut colonization by alternative techniques, including more sensitive in vivo bioluminescence detection imaging or by directly quantifying intestinal content in immunized and unimmunized animals.

This example provides evidence on how ETEC CSs are immunoprotective and emphasizes the need for a multivalent vaccine for ETEC diarrhea prevention. An ideal ETEC vaccine should include the most prevalent CSs to provide the maximum protection against highly heterogeneous ETEC populations. At least 23 specific coli surface antigens (CSs) have been identified in human ETEC strains and it is not unusual for one ETEC strain to express two or more types of CSs at the same time. Del Canto et al. 80(8):2791-2801 (2012). Although the better-performing CS21 pili conferred appreciable protection against ETEC colonization on several days post challenge, the immunized mice were still colonized with ETEC at lower levels. The inventors predict that anti-CS21 antibodies prevent ETEC binding to intestinal cell surface and ETEC floating on the mucus surface are unable to effectively deliver LT/ST toxins to cells to induce disease. The limited number of mice per group in our animal experiments may have limited the power of the study during statistical analysis. More preclinical studies with higher number of mice per group would be useful to evaluate immunoprotection against ETEC colonization.

Immunization routes and adjuvants appear to play roles in eliciting immune responses and inhibiting ETEC colonization and fecal shedding Immunizations via intraperitoneal route elicited the highest level of anti-LngA antibody responses and showed more inhibitory effects against ETEC colonization and fecal shedding. Unfortunately, intraperitoneal immunization is not practical in human vaccination. Mice immunized IN with CS21 mounted moderate antibody responses, yet, they cleared ETEC more efficiently after 7 days post challenge, indicating mucosal delivery of CSs may be important for vaccine design. IFA, a potent pro-inflammatory agent, outperformed ALUM in stimulating antibody responses and inhibiting ETEC colonization, yet ALUM, because of safety, is a preferred adjuvant in human vaccines. Aucouturier et al., Vaccine, 19(17-19):2666-72 (2001). Heat-labile enterotoxin (LT), a closely related toxin to cholera toxin, has been shown to provides a significant advantage in colonization of the small intestine in vitro and the mouse model. Allen et al., Infect. Immun 74(2):869-75 (2006). Therefore, it is reasoned that anti-LT or anti-CT immunity may promote anti-colonization immunity. Co-administration of CS21 and adjuvant CT provided significant inhibitory effects on bacteria feces shedding (FIG. 6C). This data suggest a synergistic effect on immunoprotection when CT adjuvant and CS antigens are used as immunogens. It is also likely that immune responses other than antibodies may also play roles in ETEC infection. Different antigens and adjuvant combinations, and immunization routes may elicit different immune responses profiles and, as a result, different protection outcomes. New studies should be conducted to optimize vaccine formulation and dosage, immunization route, adjuvant, and schedule to stimulate strong and protective anti-CS21 immunity. Continued research on CS21 vaccines may pave the way to vaccine research directed to the development innovative polyvalent safe and immunogenic ETEC vaccines that my reach phase I clinical trials.

In summary, LngA and CS21 elicit strong and specific systemic and mucosal antibody responses in mice. More importantly, we have demonstrated that LngA and CS21 immunization, in combination with specific adjuvants and delivered routes, inhibit ETEC colonization of the mice gut. CS21 is a highly prevalent CS among ETEC worldwide and immune responses against CS21 may contribute to protection against ETEC diarrhea in the mammalian host. Further immunoprotection preclinical studies are necessary to define the best antigen preparation, adjuvants, and delivery routes before considering CS21 phase I safety and immunogenicity clinical studies.

The complete disclosure of all patents, patent applications, and publications, and electronically available materials cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A vaccine composition comprising an LngA peptide or a fragment or variant thereof and a pharmaceutically acceptable carrier.

2. The vaccine composition of claim 3, wherein the composition comprises an LngA epitope having 90% sequence identity to an amino acid sequence selected from the group consisting of AYQRDGKYPDFV (SEQ ID NO: 1), TIKTDTSGIP (SEQ ID NO: 2), ITPDEVRNN (SEQ ID NO: 3), LTSNGAQVK (SEQ ID NO: 4), and GNNGQTTLT (SEQ ID NO: 5).

3. The vaccine composition of claim 1, wherein the composition comprises a CS21 pilus.

4. The vaccine composition of claim 1, wherein the vaccine composition is a multivalent vaccine.

5. The vaccine composition of claim 1, further comprising an adjuvant.

6. The vaccine composition of claim 4, wherein the adjuvant is cholera toxin.

7. The vaccine composition of claim 1, wherein the composition is an injectable formulation.

8. The vaccine composition of claim 1, wherein the composition is an intranasal formulation.

9. A method of preventing or inhibiting an enterotoxigenic Escherichia coli infection in a subject by administering a vaccine composition comprising an LngA peptide or a fragment or variant thereof and a pharmaceutically acceptable carrier to the subject.

10. The method of claim 9, wherein the vaccine composition comprises an LngA epitope having 90% sequence identity to an amino acid sequence selected from the group consisting of AYQRDGKYPDFV (SEQ ID NO: 1), TIKTDTSGIP (SEQ ID NO: 2), ITPDEVRNN (SEQ ID NO: 3), LTSNGAQVK (SEQ ID NO: 4), and GNNGQTTLT (SEQ ID NO: 5).

11. The method of claim 9, wherein the LngA or fragment or variant thereof of the vaccine composition is expressed by a live recombinant bacterial vector.

12. The method of claim 9, wherein the vaccine composition comprises a CS21 pilus.

13. The method of claim 9, wherein the vaccine composition further comprising an adjuvant.

14. The method of claim 13, wherein the adjuvant is cholera toxin.

15. The method of claim 9, wherein the vaccine composition is administered by injection.

16. The method of claim 9, wherein the vaccine composition is administered intranasally.

17. A method of treating infection by enterotoxigenic Escherichia coli in a subject, by administering a therapeutically effective amount of antibody that specifically binds to an LngA epitope and a pharmaceutically acceptable carrier to the subject.

18. The method of claim 17, wherein the antibody specifically binds to an LngA epitope selected from the group consisting of AYQRDGKYPDFV (SEQ ID NO: 1), TIKTDTSGIP (SEQ ID NO: 2), ITPDEVRNN (SEQ ID NO: 3), LTSNGAQVK (SEQ ID NO: 4), and GNNGQTTLT (SEQ ID NO: 5).

19. The method of claim 17, further comprising administering an antidiarrheal agent to the subject.


SEQUENCE LISTING (29 sequences)

SEQ ID NO: 1 - protein, 12 aa, Escherichia coli
AYQRDGKYPDFV

SEQ ID NO: 2 - protein, 10 aa, Escherichia coli
TIKTDTSGIP

SEQ ID NO: 3 - protein, 9 aa, Escherichia coli
ITPDEVRNN

SEQ ID NO: 4 - protein, 9 aa, Escherichia coli
LTSNGAQVK

SEQ ID NO: 5 - protein, 9 aa, Escherichia coli
GNNGQTTLT

SEQ ID NO: 6 - protein, 206 aa, Escherichia coli
MSLLEVIIVLGIIGTIAAGVVILAQRAFDSRAVTDLVTNTNTVRVRMKDAYQRDGKYPDFVDPLSLTANTIKTDTSGIPAAQLVQLGKITPDEVRNNISGDFIAIGGALTSNGAQVKKGFAIELNGLSQEQCRSILGQVGNNWEYVAIGTSASGSYAMTATGVDMSVAASTTVLRSLGNNGQTTLTADKILSTCTAQVNSITLGSR

SEQ ID NO: 7 - protein, 6 aa, Artificial Sequence
  Description of Artificial Sequence Synthetic 6xHis tag
HHHHHH

SEQ ID NO: 8 - protein, 206 aa, Escherichia coli
MSLLEVIIVLGIIGTIAAGVVILAQRAFDSRAVTDLVTNTNTVRVAMKDAYQRDGKYPDFVDPLSLTANTIKTDTNGIPAAQLVQLGKITPDEVRNNISGDFIAIGGALTSNGAQVKKGFAIELNGLSQEQCRSILGQVGNNWEYVAIGTSASGSYAMTATGVDMSVAASTTVLRSLGNNGQTTLTADKILSTCTAQVNSITLGSR

SEQ ID NO: 9 - protein, 206 aa, Escherichia coli
MSLLEVIIVLGIIGTIAAGVVILAQRAFDSRAVTDLVTNTNTVRVAMKDAYQRDGVYPDYVSPLNLTSDTIKTNTGKIPAAQLVQLGKITPDEVRNNISGDFIAIGGALTSNSATVKKGFAIELNGLSQEQCRSILGQVGNNWEYVAVGNSASGSYAMQAAGVDMSVAAAGDILRTLGTDGQKTLTAEKILGTCTATVNSITLGSR

SEQ ID NO: 10 - protein, 206 aa, Escherichia coli
MSLLEVIIVLGIIGTIAAGVVILAQRAFDSRAVTDLVTNTNTVRVAMKDAYQRDGVYPDYVSPLNLTSDTIKTDTGKIPAAQLVQLGKITPDEVRNNISGDFIAIGGALTSDGATVKKGFAIELNGLSQEQCRSILGQVGNNWEYVAVGNSASGSYAMQTAGVNMSVAATGNILRTLGTDGQTTLTADKILSTCTAQVNSITLGSR

SEQ ID NO: 11 - protein, 206 aa, Escherichia coli
MSLLEVIIVLGIIGTIAAGVVILAQRAFDSRAVTDLVTNTNTVRVAMKDAYQRDGVYPDYVSPLNLTSDTIKTDTGKIPAAQLVQLGKITPDEVRNNISGDFIAIGGALTSDGATVKKGFAIELNGLSQEQCRSILGQVGNNWEYVAVGNSASGSYAMQTAGVNMSVAATGNILRTLGTDGQKTLTAEKILGTCTATVNSITLGSR

SEQ ID NO: 12 - protein, 206 aa, Escherichia coli
MSLLEVIIVLGIIGTIAAGVVILAQRAFDSRAVTDLVTNTNTVRVAMKDAYQRDGKYPDFVDPLSLTANTIKTDTSGIPAAQLVQLGKITPDEVRNNISGDFIAIGGALTSNGAQVKKGFAIELNGLSQEQCRSILGQVGNNWEYVAIGTSASGSYAMTATGVDMSVAASTTVLRSLGNGGQTTLTADKILSTCTAQVNSITLGSR

SEQ ID NO: 13 - protein, 206 aa, Escherichia coli
MSLLEVIIVLGIIGTIAAGVVILAQRAFDSRAVTDLVTNTNTVRVAMKDAYQRDGVYPDYVSPLNLTSDTIKTDTGKIPAAQLVQLGKITPDEVRNNISGDFIAIGGALTSDGATVKKGFAIELNGLSQEQCRSILGQVGNNWEYVAVGNSASGSYAMQTAGVNMSVAATGNILRTLGTDGQKTLTAERILGTCTATVNSITLGSR

SEQ ID NO: 14 - protein, 206 aa, Escherichia coli; identical to SEQ ID NO: 6

SEQ ID NO: 15 - protein, 206 aa, Escherichia coli; identical to SEQ ID NO: 13

SEQ ID NO: 16 - protein, 206 aa, Escherichia coli
MSLLEVIIVLGIIGTIAAGVVILAQRAFDSRAVTDLVTNTNTVRVAMKDAYQRDGVYPDYVSPLKLTSDTIKKDTGKIPAAQLVQLGKITPDEVRNNISGDFIAIGGALTSNGATVKKGFAIELNGLSQEQCRSILGQVGNNWEYVAVGNSASGSYAMQAAEVDMSVAATGDILRTLGTDGQTTLTADKILSTCTAQVNSITLGSR

SEQ ID NO: 17 - protein, 206 aa, Escherichia coli
MSLLEVIIVLGIIGTIAAGVVILAQRAFDSRAVTDLVTNTNTVRVAMKDAYQRDGVYPDYVSPLKLTSDTIKKDTGKIPAAQLVQLGKITPDEVRNNISGDFIAIGGALTSNGATVKKGFAIELNGLSQEQCRSILGQVGNNWEYVAVGNSASGSYAMQAAEVDMSVAATGDILRTLGTDGQKTLTAERILGTCTATVNSITLGSR

SEQ ID NO: 18 - protein, 206 aa, Escherichia coli; identical to SEQ ID NO: 8

SEQ ID NO: 19 - protein, 206 aa, Escherichia coli
MSLLEVIIVLGIIGTIAAGVVILAQRAFDSRAVTDLVTNTNTVRVAMKDAYQRDGVYPDYVSPLNLTSDTIKTDTGKIPAAQLVQLGKITPDEVRNNISGDFIAIGGALTSDGATVKKGFAIELNGLSQEQCRSILGQVGNNWEYVAVGNSASGSYAMQTAGVNMSVAATGNILRTLGTDGQTTLTADKILRTCTAQVNSITLGSR

SEQ ID NO: 20 - protein, 206 aa, Escherichia coli; identical to SEQ ID NO: 17

SEQ ID NO: 21 - protein, 206 aa, Escherichia coli; identical to SEQ ID NO: 8

SEQ ID NO: 22 - protein, 206 aa, Escherichia coli; identical to SEQ ID NO: 11

SEQ ID NO: 23 - protein, 206 aa, Escherichia coli; identical to SEQ ID NO: 8

SEQ ID NO: 24 - protein, 206 aa, Escherichia coli; identical to SEQ ID NO: 8

SEQ ID NO: 25 - protein, 206 aa, Escherichia coli; identical to SEQ ID NO: 8

SEQ ID NO: 26 - protein, 206 aa, Escherichia coli; identical to SEQ ID NO: 17

SEQ ID NO: 27 - protein, 199 aa, Vibrio cholerae
MTLLEVIIVLGIMGVVSAGVVTLAQRAIDSQNMTKAAQNLNSVQIAMTQTYRSLGNYPATANANAATQLANGLVSLGKVSADEAKNPFTGTAMGIFSFPRNSAANKAFAITVGGLTQAQCKTLVTSVGDMFPFINVKEGAFAAVADLGDFETSVADAATGAGVIKSIAPGSANLNLTNITHVEKLCTGTAPFTVAFGNS

SEQ ID NO: 28 - protein, 205 aa, Escherichia coli
MSLLEVIIVLGIIGTIAAGVVILAQRAFDSRTVSELVTNTNTIRVAMKDAYQRDGKYPDYADPLALTADTIKTGNSTIPVAQLVQLGKLTPDEARNGISGDYIGIGGAKTSNSNVNKGFVIELNGLSQEQCRSILGQVGNNWEFVEVGTSGSGSYSITAQVDMTKESGNNTTLRSLGLNGQNSITSSSILNTCTATVNSIILGSR

SEQ ID NO: 29 - protein, 193 aa, Escherichia coli
MVSKIMNKKYEKGLSLIESAMVLALAATVTAGVMFYYQSASDSNKSQNAISEVMSATSAINGLYIGQTSYSGLDSTILLNTSAIPDNYKDTTNKKITNPFGGELNVGPANNNTAFGYYLTLTRLDKAACVSLATLNLGTSAKGYGVNISSENNITSFGNSADQAAKSTAITPAEAATACKNTDSTNKVTYFMK